(19) United States
(12) Patent Application Publication
(10) Pub. No.: US 2013/0345312 A1
(43) Pub. Date: Dec. 26, 2013

(54) TREATMENT OF MITOCHONDRIAL DISEASES WITH NAPHTHOQUINONES

(71) Applicants: Orion D. Jankowski, Burlingame, CA (US); Andrew W. Hinman, San Francisco, CA (US); Guy M. Miller, Monte Sereno, CA (US)

(72) Inventors: Orion D. Jankowski, Burlingame, CA (US); Andrew W. Hinman, San Francisco, CA (US); Guy M. Miller, Monte Sereno, CA (US)

(21) Appl. No.: 13/814,721

(22) PCT Filed: Aug. 4, 2011
(86) PCT No.: PCT/US2011/046630

§ 371 Date: Jun. 4, 2013

Related U.S. Application Data
(60) Provisional application No. 61/401,044, filed on Aug. 6, 2010.

(51) Int. Cl. C07C 50/32

(52) U.S. Cl. CPC C07C 50/32
USPC 514/682

(57) ABSTRACT

Methods of treating, preventing or suppressing symptoms associated with mitochondrial diseases, such as Friedreich's ataxia (FRDA), Leber's Hereditary Optic Neuropathy (LHON), dominant optic atrophy (DOA); mitochondrial myopathy, encephalopathy, lactacidosis, stroke (MELAS), Leigh syndrome or Kearns-Sayre Syndrome (KSS) with compounds of Formula (I) are disclosed. Methods of modulating, normalizing, or enhancing energy biomarkers, as well as compounds useful for such methods are also disclosed.

### CROSS-REFERENCE TO RELATED APPLICATIONS

This application claims priority benefit of U.S. Provisional Patent Application No. 61/401,044, filed Aug. 6, 2010. The entire content of that application is hereby incorporated by reference herein.

### TECHNICAL FIELD OF THE INVENTION

The application discloses compositions and methods useful for treatment, prevention, or suppression of diseases due to mitochondrial disorders such as Friedreich's ataxia; Leber's Hereditary Optic Neuropathy; dominant optic atrophy; Kearns-Sayre Syndrome; Leigh syndrome; and MELAS; and for modulating energy biomarkers with naphthoquinones of Formula I in a subject in need of such treatment. This application does not relate to naphthoquinones commonly called Vitamin K.

### BACKGROUND

Mitochondria are organelles in eukaryotic cells, popularly referred to as the “powerhouse” of the cell. One of their primary functions is oxidative phosphorylation. The molecule adenosine triphosphate (ATP) functions as an energy “currency” or energy carrier in the cell, and eukaryotic cells derive the majority of their ATP from biochemical processes carried out by mitochondria. These biochemical processes include the citric acid cycle (the tricarboxylic acid cycle, or Krebs cycle), which generates reduced nicotinamide adenine dinucleotide (NADH+H⁺) from oxidized nicotinamide adenine dinucleotide (NAD⁺), and oxidative phosphorylation, during which NADH+H⁺ is oxidized back to NAD⁺. (The citric acid cycle also reduces flavin adenine dinucleotide, or FAD, to FADH₂; FADH₂ also participates in oxidative phosphorylation.)

The electrons released by oxidation of NADH+H⁺ are shuttled down a series of protein complexes (Complex I, Complex II, Complex III, and Complex IV) known as the mitochondrial respiratory chain. These complexes are embedded in the inner membrane of the mitochondrion. Complex IV, at the end of the chain, transfers the electrons to oxygen, which is reduced to water. The energy released as these electrons traverse the complexes is used to generate a proton gradient across the inner membrane of the mitochondrion, which creates an electrochemical potential across the inner membrane. Another protein complex, Complex V (which is not directly associated with Complexes I, II, III and IV) uses the energy stored by the electrochemical gradient to convert ADP into ATP.

Mitochondrial dysfunction contributes to various disease states. Some mitochondrial diseases are due to mutations or deletions in the mitochondrial genome. If a threshold proportion of mitochondria in the cell is defective, and if a threshold proportion of such cells within a tissue have defective mitochondria, symptoms of tissue or organ dysfunction can result. Practically any tissue can be affected, and a large variety of symptoms may be present, depending on the extent to which different tissues are involved. Some examples of mitochondrial diseases are Friedreich's ataxia (FRDA), Leber's Hereditary Optic Neuropathy (LHON), dominant optic atrophy (DOA), mitochondrial myopathy, encephalopathy, lactacidosis, and stroke (MELAS), Myoclonus Epilepsy Associated with Ragged-Red Fibers (MERRF) syndrome, Leigh syndrome, and respiratory chain disorders. Most mitochondrial diseases involve children who manifest the signs and symptoms of accelerated aging, including neurodegenerative diseases, stroke, blindness, hearing impairment, diabetes, and heart failure.

Friedreich's ataxia is an autosomal recessive neurodegenerative and cardiodegenerative disorder caused by decreased levels of the protein Frataxin. The disease causes the progressive loss of voluntary motor coordination (ataxia) and cardiac complications. Symptoms typically begin in childhood, and the disease progressively worsens as the patient grows older; patients eventually become wheelchair-bound due to motor disabilities.

Leber's Hereditary Optic Neuropathy (LHON) is a disease characterized by blindness which occurs on average between 27 and 34 years of age. Other symptoms may also occur, such as cardiac abnormalities and neurological complications.

Mitochondrial myopathy, encephalopathy, lactacidosis, and stroke (MELAS) can manifest itself in infants, children, or young adults. Strokes, accompanied by vomiting and seizures, are one of the most serious symptoms; it is postulated that the metabolic impairment of mitochondria in certain areas of the brain is responsible for cell death and neurological lesions, rather than the impairment of blood flow as occurs in ischemic stroke.

Myoclonus Epilepsy Associated with Ragged-Red Fibers (MERRF) syndrome is one of a group of rare muscular disorders that are called mitochondrial encephalomyopathies. Mitochondrial encephalomyopathies are disorders in which a defect in the genetic material arises from a part of the cell structure that releases energy (mitochondria). This can cause a dysfunction of the brain and muscles (encephalomyopathies). The mitochondrial defect as well as “ragged-red fibers” (an abnormality of tissue when viewed under a microscope) are always present. The most characteristic symptom of MERRF syndrome is myoclonic seizures that are usually sudden, brief, jerking, spasms that can affect the limbs or the entire body. Difficulty speaking (dysarthria), optic atrophy, short stature, hearing loss, dementia, and involuntary jerking of the eyes (nystagmus) may also occur.

Leigh syndrome is a rare inherited neurometabolic disorder characterized by degeneration of the central nervous system where the symptoms usually begin between the ages of 3 months to 2 years and progress rapidly. In most children, the first signs may be poor sucking ability and loss of head control and motor skills. These symptoms may be accompanied by loss of appetite, vomiting, irritability, continuous crying, and seizures. As the disorder progresses, symptoms may also include generalized weakness, lack of muscle tone, and episodes of lactic acidosis, which can lead to impairment of respiratory and kidney function. Heart problems may also occur. Leigh syndrome arises from mutations that affect Complex IV. These mutations include mitochondrial-encoded MTCO3; nuclear-encoded COX10, COX15, SCO2, SURF1, which is involved in the assembly of complex IV, and TACO1; see www.ncbi.nlm.nih.gov/entrez/dispomim.cgi?id=256000.

Co-Enzyme Q10 Deficiency is a respiratory chain disorder, with syndromes such as myopathy with exercise intolerance and recurrent myoglobin in the urine manifested by ataxia, seizures or mental retardation and leading to renal failure (Di Mauro et al., (2005) Neuromusc. Disord., 15:311-315), childhood-onset cerebellar ataxia and cerebellar atrophy (Masumeci et al., (2001) Neurology 56:849-855 and Lamperti et al., (2003) Neurology 60:1206:1208); and infantile encephalomyopathy associated with nephrosis. Biochemical measurement of muscle homogenates of patients with CoQ10 deficiency showed severely decreased activities of respiratory chain complexes I and II+III, while complex IV (COX) was moderately decreased (Gempel et al., (2007) Brain, 130(8):2037-2044).

Complex I Deficiency or NADH dehydrogenase NADH-CoQ reductase deficiency is a respiratory chain disorder, with symptoms classified by three major forms: (1) fatal infantile multisystem disorder, characterized by developmental delay, muscle weakness, heart disease, congenital lactic acidosis, and respiratory failure; (2) myopathy beginning in childhood or in adult life, manifesting as exercise intolerance or weakness; and (3) mitochondrial encephalomyopathy (including MELAS), which may begin in childhood or adult life and consists of variable combinations of symptoms and signs, including ophthalmoplegia, seizures, dementia, ataxia, hearing loss, pigmentary retinopathy, sensory neuropathy, and uncontrollable movements.

Complex II Deficiency or Succinate dehydrogenase deficiency is a respiratory chain disorder with symptoms including encephalomyopathy and various manifestations, including failure to thrive, developmental delay, hypotonia, lethargy, respiratory failure, ataxia, myoclonus and lactic acidosis.

Complex III Deficiency or Ubiquinone-cytochrome C oxidoreductase deficiency is a respiratory chain disorder with symptoms categorized in four major forms: (1) fatal infantile encephalomyopathy, congenital lactic acidosis, hypotonia, dystrophic posturing, seizures, and coma; (2) encephalomyopathies of later onset (childhood to adult life): various combinations of weakness, short stature, ataxia, dementia, hearing loss, sensory neuropathy, pigmentary retinopathy, and pyramidal signs; (3) myopathy, with exercise intolerance evolving into fixed weakness; and (4) infantile histiocytoid cardiomyopathy.

Complex IV Deficiency or Cytochrome C oxidase deficiency is a respiratory chain disorder with symptoms categorized in two major forms: (1) encephalomyopathy, which is typically normal for the first 6 to 12 months of life and then show developmental regression, ataxia, lactic acidosis, optic atrophy, ophthalmoplegia, nystagmus, dystonia, pyramidal signs, respiratory problems and frequent seizures; and (2) myopathy with two main variants: (a) Fatal infantile myopathy-may begin soon after birth and accompanied by hypotonia, weakness, lactic acidosis, ragged-red fibers, respiratory failure, and kidney problems: and (b) benign infantile myopathy—may begin soon after birth and accompanied by hypotonia, weakness, lactic acidosis, ragged-red fibers, respiratory problems, but (if the child survives) followed by spontaneous improvement.

Complex V Deficiency or ATP synthase deficiency is a respiratory chain disorder including symptoms such as slow, progressive myopathy.

CPEO or Chronic Progressive External Ophthalmoplegia Syndrome is a respiratory chain disorder including symptoms such as visual myopathy, retinitis pigmentosa, or dysfunction of the central nervous system.

Kearns-Sayre Syndrome (KSS) is a mitochondrial disease characterized by a triad of features including: (1) typical onset in persons younger than age 20 years; (2) chronic, progressive, external ophthalmoplegia; and (3) pigmentary degeneration of the retina. In addition, KSS may include cardiac conduction defects, cerebellar ataxia, and raised cerebrospinal fluid (CSF) protein levels (e.g., >100 mg/dL). Additional features associated with KSS may include myopathy, dystonia, endocrine abnormalities (e.g., diabetes, growth retardation or short stature, and hypoparathyroidism), bilateral sensorineural deafness, dementia, cataracts, and proximal renal tubular acidosis.

In addition to congenital disorders involving inherited defective mitochondria, acquired mitochondrial dysfunction contributes to diseases, particularly neurodegenerative disorders associated with aging like Parkinson's, Alzheimer's, and Huntington's Diseases. The incidence of somatic mutations in mitochondrial DNA rises exponentially with age; diminished respiratory chain activity is found universally in aging people. Mitochondrial dysfunction is also implicated in excitoxic, neuronal injury, such as that associated with cerebral vascular accidents, seizures and ischemia.

Recent studies have suggested that as many 20 percent of patients with autism have markers for mitochondrial disease, (Shoffner, J. the 60^{th} Annual American Academy of Neurology meeting in Chicago, Apr. 12-19, 2008; Poling, J S et al J. child Neurol. 2008, 21(2) 170-2; and Rossignol et al., Am. J. Biochem. & Biotech. (2008)₄, 208-217.)

Genetic mitochondrial mutations have also been correlated to hearing loss. This has been demonstrated by the presence of mitochondrial DNA mutations in families with non-syndromic progressive sensorineural hearing loss (SNHL) (Berretinin, S. et al., Biosci. Rep. (2008) 28. 45-59; and Devarjan et al. Hearing Research, (2002) 174, 45-54) suggest involvement of mitochondrial pathways in cisplatin-induced apoptosis in a model in vitro system of cultured auditory cells.

Very few treatments are available for patients suffering from these mitochondrial diseases. Recently, the compound Idebenone has been proposed for treatment of Friedreich's ataxia. While the clinical effects of Idebenone have been relatively modest, the complications of mitochondrial diseases can be so severe that even marginally useful therapies are preferable to the untreated course of the disease. Another compound, MitoQ, has been proposed for treating mitochondrial disorders (see U.S. Pat. No. 7,179,928); clinical results for MitoQ have not yet been reported. Administration of coenzyme Q10 (CoQ10) and vitamin supplements has shown only transient beneficial effects in individual cases of KSS. CoQ10 supplementation has also been used for the treatment of CoQ10 deficiency with mixed results.

The ability to adjust biological production of energy has applications beyond the diseases described above. Various other disorders can result in suboptimal levels of energy biomarkers (sometimes also referred to as indicators of energetic function), such as ATP levels. Treatments for these disorders are also needed, in order to modulate one or more energy biomarkers to improve the health of the patient. In other applications, it can be desirable to modulate certain energy biomarkers away from their normal values in an individual that is not suffering from disease. For example, if an individual is undergoing an extremely strenuous undertaking, it can be desirable to raise the level of ATP in that individual.

Accordingly, there is a serious and unmet need for effective treatments of mitochondrial disorders.

### DISCLOSURE OF THE INVENTION

The invention embraces methods of treatment, prevention, or suppression of symptoms associated with a mitochondrial disorder, modulating one or more energy biomarkers, normalizing one or more energy biomarkers, or enhancing one or more energy biomarkers, comprising administering to a subject a therapeutically effective amount or effective amount of one or more compounds as described herein.

In one embodiment, the invention embraces a method of treating a mitochondrial disorder, modulating one or more energy biomarkers, normalizing one or more energy biomarkers, or enhancing one or more energy biomarkers, comprising administering to a subject an effective amount of one or more compounds of Formula I:

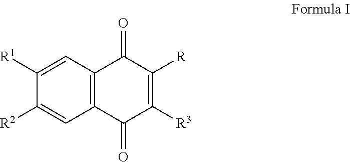

wherein,
R is selected from the group consisting of hydrogen; —O(C₁-C₆)alkyl; —(CH₂)₀₋₁₉—CH₃; —((CH₂)₂—CH(CH₃))₁₋₂₀—CH₃;

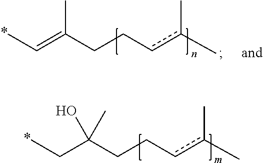

the * indicates the point of attachment to R;
the bond indicated by a dashed line is independently in each occurrence double or single and each unit can be the same or different;
R¹, R² and R³ are independently of each other hydrogen; —(C₁-C₆)alkyl; or —O(C₁-C₆)alkyl;
n is 0-12, wherein when n is 2-12 each unit can be the same or different; and
m is 1-12, wherein when m is 2-12 each unit can be the same or different;
with the proviso that when R¹ and R² are hydrogen, and R³ is —(C₁-C₆)alkyl, then R is not

hydrogen or

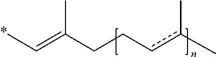

or any stereoisomer, mixture of stereoisomers, prodrug, metabolite, salt, crystalline form, non-crystalline form, hydrate or solvate thereof.

In another embodiment, the invention embraces a method of treating a mitochondrial disorder, modulating one or more energy biomarkers, normalizing one or more energy biomarkers, or enhancing one or more energy biomarkers, comprising administering to a subject an effective amount of one or more compounds of Formula I wherein:

R is selected from the group consisting of —(CH₂)₀₋₁₉—CH₃, —((CH₂)₂—CH(CH₃))₁₋₂₀—CH₃;

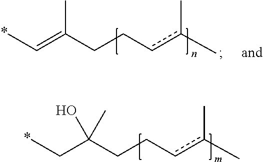

the * indicates the point of attachment to R;
the bond indicated by a dashed line is independently in each occurrence double or single, where each unit can be the same or different;
R¹ and R² are independently of each other hydrogen; —(C₁-C₆)alkyl; or —O(C₁-C₆)alkyl;
R³ is hydrogen or —(C₁-C₆)alkyl;
n is 0-12, wherein when n is 2-12 each unit can be the same or different; and
m is 1-12, wherein when m is 2-12 each unit can be the same or different;
with the proviso that when R¹ and R² are hydrogen, and R³ is —(C₁-C₆)alkyl, then R is not hydrogen or

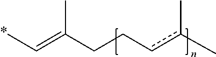

or any stereoisomer, mixture of stereoisomers, prodrug, metabolite, salt, crystalline form, non-crystalline form, hydrate or solvate thereof.

In one embodiment, R¹, R² and R³ are independently of each other —(C₁-C₆)alkyl. In another embodiment, R¹, R² and R³ are methyl. In another embodiment, R¹ and R² are hydrogen and R³ is —(C₁-C₆)alkyl. In another embodiment, R¹ and R² are hydrogen and R³ is methyl. In another embodiment, R¹ and R² are independently of each other —O(C₁-C₆)alkyl and R³ is —(C₁-C₆)alkyl. In another embodiment, R¹ and R² are methoxy and R³ is methyl. In some embodiments, m is 1. In some embodiments, n is 1. In other embodiments, m is 2. In some embodiments, n is 2. In other embodiments, m is 3. In some embodiments, n is 3. In other embodiments, m is 4. In some embodiments, n is 4. In other embodiments, m is 5. In some embodiments, n is 5. In other embodiments, m is 6. In some embodiments, n is 6. In other embodiments, m is 7. In some embodiments, n is 7. In other embodiments, m is 8. In some embodiments, n is 8. In other embodiments, m is 9. In some embodiments, n is 9. In other embodiments, m is 10. In some embodiments, n is 10. In other embodiments, m is 11. In some embodiments, n is 11. In other embodiments, m is 12. In some embodiments, n is 12.

In another embodiment, R¹ and R² are hydrogen and R and R³ are —(C₁-C₆)alkyl. In another embodiment, R¹ and R² are hydrogen and R and R³ are methyl. In another embodiment, R, R¹ and R² are hydrogen and R³ is —O(C₁-C₆)alkyl. In another embodiment, R, R¹ and R² are hydrogen and R³ is methoxy. In another embodiment, R, R′, R², and R³ are hydrogen. In another embodiment, R¹ and R² are hydrogen and R and R³ are —O(C₁-C₆)alkyl. In another embodiment, R¹ and R² are hydrogen and R and R³ are methoxy.

In one embodiment, the invention embraces a method of treating, preventing, or suppressing symptoms associated with a mitochondrial disorder, modulating one or more energy biomarkers, normalizing one or more energy biomarkers, or enhancing one or more energy biomarkers, comprising administering to a subject an effective amount of one or more compounds of Formula I, wherein at least one of the compounds is a compound of Formula Ia:

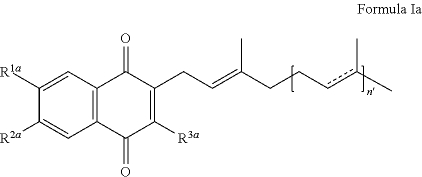

wherein,
the bond indicated by a dashed line can be independently in each occurrence double or single and where each unit can be the same or different;
R^{1a} and R^{2a} are independently of each other, —(C₁-C₆)alkyl or —O(C₁-C₆)alkyl;
R^{3a} is hydrogen or —(C₁-C₆)alkyl;
n′ is 0-12, wherein when n′ is 2-12 each unit can be the same or different; or any stereoisomer, mixture of stereoisomers, prodrug, metabolite, salt, crystalline form, non-crystalline form, hydrate or solvate thereof.

In one embodiment, R^{1a}, R^{2a} and R^{3a} are independently of each other —(C₁-C₆)alkyl. In another embodiment, R^{1a}, R^{2a} and R^{3a} are methyl. In another embodiment, R^{1a} and R^{2a} are independently of each other —O(C₁-C₆)alkyl and R^{3a} is —(C₁-C₆)alkyl. In another embodiment, R^{1a} and R^{2a} are independently of each other methoxy and R^{3a} is methyl.

In another embodiment, R^{1a} and R^{2a} are methoxy and R^{3a} is methyl. In another embodiment, R^{1a} and R^{2a} are independently of each other —O(C₁-C₆)alkyl and R^{3a} is hydrogen. In another embodiment, R^{1a} and R^{2a} are independently of each other methoxy and R^{3a} is hydrogen. In some embodiments, n′ is 1. In other embodiments, n′ is 2. In other embodiments n′ is 3. In other embodiments, n′ is 4. In other embodiments, n′ is 5. In other embodiments, n′ is 6. In other embodiments n′ is 7. In other embodiments, n′ is 8. In other embodiments, n′ is 9. In other embodiments, n′ is 10. In other embodiments, n′ is 11. In other embodiments, n′ is 12. In another embodiment, the bond indicated with a dashed line is a single bond in every unit. In another embodiment, the bond indicated with a dashed line is a double bond in every unit.

The invention does not relate to Vitamin K compounds. In all embodiments, the compound of Formula I is not a Vitamin K2 compound. In all embodiments, the compound of Formula I is not selected from vitamin MK-2, vitamin MK-3, vitamin MK-4, vitamin MK-5, vitamin MK-6, vitamin MK-7, vitamin MK-8, vitamin MK-9, vitamin MK-10, vitamin MK-11, vitamin MK-12 and vitamin MK-13. In all embodiments the compound of Formula I wherein the bond indicated with a dashed line is a single bond, is not Vitamin K1 also known as Phylloquinone.

In some embodiments, the invention embraces a method of treating a mitochondrial disorder, modulating one or more energy biomarkers, normalizing one or more energy biomarkers, or enhancing one or more energy biomarkers, comprising administering to a subject an effective amount of the compound of Formula Ia, with the proviso that said compound is not Vitamin K or any forms thereof.

In some embodiments, the invention additionally embraces a method of treating a mitochondrial disorder, modulating one or more energy biomarkers, normalizing one or more energy biomarkers, or enhancing one or more energy biomarkers, comprising administering to a subject an effective amount of one or more compounds of Formula I or Formula Ia selected from:
6,7-dimethoxy-2-methyl-3-(3,7,11,15-tetramethylhexadec-2-en-1-yl)naphthalene-1,4-dione;
2-(3,7-dimethyloct-2-en-1-yl)-6,7-dimethoxy-3-methylnaphthalene-1,4-dione;
2-(3,7-dimethyloct-2-en-1-yl)-3,6,7-trimethylnaphthalene-1,4-dione;
2,6,7-trimethyl-3-(3,7,11,15-tetramethylhexadeca-2,6-dien-1-yl)naphthalene-1,4-dione;
6,7-dimethoxy-2-methyl-3-(3,7,11,15-tetramethylhexadeca-2,6-dien-1-yl)naphthalene-1,4-dione;
2-(3,7-dimethylocta-2,6-dien-1-yl)-3,6,7-trimethylnaphthalene-1,4-dione;
2,6,7-trimethyl-3-(3,7,11-trimethyldodeca-2,6,10-trien-1-yl)naphthalene-1,4-dione;
2,6,7-trimethyl-3-3,7,11,15-tetramethylhexadeca-2,6,10,14-tetraen-1-yl)naphthalene-1,4-dione;
2,6,7-trimethyl-3-(3,7,11,15,19-pentamethylicosa-2,6,10,14,18-pentaen-1-yl)naphthalene-1,4-dione;
2-(−3,7,11,15,19,23-hexamethyltetracosa-2,6,10,14,18,22-hexaen-1-yl)-3,6,7-trimethylnaphthalene-1,4-dione;
2-(−3,7,11,15,19,23,27-heptamethyloctacosa-2,6,10,14,18,22,26-heptaen-1-yl)-3,6,7-trimethylnaphthalene-1,4-dione;
2-(3,7-dimethylocta-2,6-dien-1-yl)-6,7-dimethoxy-3-methylnaphthalene-1,4-dione;
6,7-dimethoxy-2-methyl-3-(3,7,11-trimethyldodeca-2,6,10-trien-1-yl)naphthalene-1,4-dione;
6,7-dimethoxy-2-methyl-3-(3,7,11,15-tetramethylhexadeca-2,6,10,14-tetraen-1-yl)naphthalene-1,4-dione;
6,7-dimethoxy-2-methyl-3-(3,7,11,15,19-pentamethylicosa-2,6,10,14,18-pentaen-1-yl)naphthalene-1,4-dione; and
2-(3,7,11,15,19,23-hexamethyltetracosa-2,6,10,14,18,22-hexaen-1-yl)-6,7-dimethoxy-3-methylnaphthalene-1,4-dione;
   or any stereoisomer, mixture of stereoisomers, prodrug, metabolite, salt, crystalline form, non-crystalline form, hydrate or solvate thereof.

In another embodiment, the invention embraces a method of treating a mitochondrial disorder, modulating one or more energy biomarkers, normalizing one or more energy biomarkers, or enhancing one or more energy biomarkers, comprising administering to a subject an effective amount of one or more compounds of Formula I, wherein at least one of the compounds is a compound of Formula Ib:

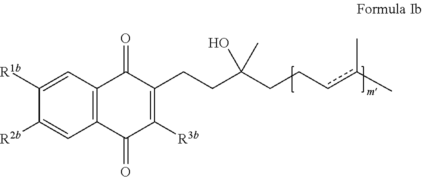

the bond indicated by a dashed line is independently in every occurrence double or single and where each unit is the same or different;
R^{1b} and R^{2b} are independently of each other hydrogen; —(C₁-C₆)alkyl; or —O(C₁-C₆)alkyl;
R^{3b} is hydrogen or —(C₁-C₆)alkyl;
m′ is 1-12, wherein when m′ is 2-12 each unit can be the same or different; or any stereoisomer, mixture of stereoisomers, prodrug, metabolite, salt, crystalline form, non-crystalline form, hydrate or solvate thereof.

In one embodiment, R^{1b}, R^{2b} and R^{3b} are independently of each other —(C₁-C₆)alkyl. In another embodiment, R^{1b}, R^{2b} and R^{3b} are methyl. In another embodiment, R^{1b} and R^{2a} are hydrogen and R^{3b} is —(C₁-C₆)alkyl. In another embodiment, R^{1b} and R^{2b} are hydrogen and R^{3b} is methyl. In another embodiment, R^{1b} and R^{2b} are independently of each other —O(C₁-C₆)alkyl and R^{3b} is —(C₁-C₆)alkyl. In another embodiment, R^{1b} and R^{2b} are independently of each other methoxy and R^{3b} is methyl. In some embodiments, m′ is 1. In other embodiments, m′ is 2. In other embodiments, m′ is 3. In other embodiments, m′ is 4. In other embodiments, m′ is 5. In other embodiments, m′ is 6. In other embodiments, m′ is 7. In other embodiments, m′ is 8. In other embodiments, m′ is 9. In other embodiments, m′ is 10. In other embodiments, m′ is 11. In other embodiments, m′ is 12. In another embodiment, the bond indicated by a dashed line is a double bond in every unit. In another embodiment the bond indicated by a dashed line is a single bond in every unit.

In some embodiments, the invention embraces a method of treating a mitochondrial disorder, modulating one or more energy biomarkers, normalizing one or more energy biomarkers, or enhancing one or more energy biomarkers, comprising administering to a subject an effective amount of the compound of Formula I or Formula Ib wherein said compound is 2-(3-hydroxy-3,7,11,15-tetramethylhexadeca-6,10,14-trien-1-yl)-3-methylnaphthalene-1,4-dione with the following formula:

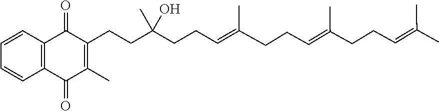 [SMILES: CC(C)=CCC/C(C)=C/CC/C(C)=C/CCC(C)(O)CCC1=C(C)C(=O)c2ccccc2C1=O]

or any stereoisomer, mixture of stereoisomers, prodrug, metabolite, salt, crystalline form, non-crystalline form, hydrate or solvate thereof.

In some embodiments, the invention embraces a method of treating a mitochondrial disorder, modulating one or more energy biomarkers, normalizing one or more energy biomarkers, or enhancing one or more energy biomarkers, comprising administering to a subject an effective amount of the compound of Formula I or Formula Ib wherein said compound is 2-(3-hydroxy-3,7,11,15-tetramethylhexadeca-6,10,14-trien-1-yl)naphthalene-1,4-dione:

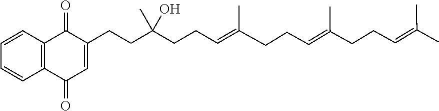 [SMILES: CC(C)=CCC/C(C)=C/CC/C(C)=C/CCC(C)(O)CCC1=CC(=O)c2ccccc2C1=O]

or any stereoisomer, mixture of stereoisomers, prodrug, metabolite, salt, crystalline form, non-crystalline form, hydrate or solvate thereof.

In some embodiments, the invention embraces a method of treating a mitochondrial disorder, modulating one or more energy biomarkers, normalizing one or more energy biomarkers, or enhancing one or more energy biomarkers, comprising administering to a subject an effective amount of the compound of Formula I or Formula Ib wherein said compound is 2-(3-hydroxy-3,7,11,15-tetramethylhexadeca-6,10,14-trien-1-yl)-3,6,7-trimethylnaphthalene-1,4-dione:

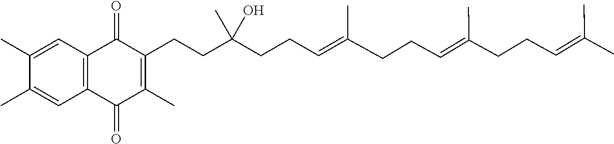 [SMILES: CC(C)=CCC/C(C)=C/CC/C(C)=C/CCC(C)(O)CCC1=C(C)C(=O)c2cc(C)c(C)cc2C1=O]

or any stereoisomer, mixture of stereoisomers, prodrug, metabolite, salt, crystalline form, non-crystalline form, hydrate or solvate thereof.

In some embodiments, the invention embraces a method of treating a mitochondrial disorder, modulating one or more energy biomarkers, normalizing one or more energy biomarkers, or enhancing one or more energy biomarkers, comprising administering to a subject an effective amount of the compound of Formula I or Formula Ib wherein said compound is 2-(3-hydroxy-3,7,11,15-tetramethylhexadeca-6,10,14-trien-1-yl)-6,7-dimethoxy-3-methylnaphthalene-1,4-dione:

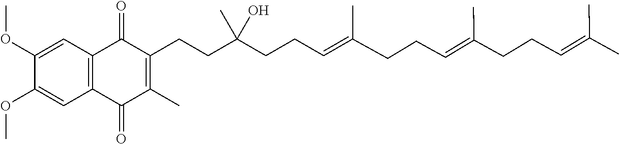 [SMILES: COc1cc2c(cc1OC)C(=O)C(CCC(C)(O)CC/C=C(\C)CC/C=C(\C)CCC=C(C)C)=C(C)C2=O]

or any stereoisomer, mixture of stereoisomers, prodrug, metabolite, salt, crystalline form, non-crystalline form, hydrate or solvate thereof.

In some embodiments, the invention additionally embraces a method of treating a mitochondrial disorder, modulating one or more energy biomarkers, normalizing one or more energy biomarkers, or enhancing one or more energy biomarkers, comprising administering to a subject an effective amount of one or more compounds of Formula I or Formula Ib selected from:
2-(3-hydroxy-3,7,11,15-tetramethylhexadecyl)naphthalene-1,4-dione;
2-(3-hydroxy-3,7,11,15-tetramethylhexadecyl)-3-methylnaphthalene-1,4-dione;
2-(3-hydroxy-3,7,11,15-tetramethylhexadecyl)-3,6,7-trimethylnaphthalene-1,4-dione;
2-(3-hydroxy-3,7,11,15-tetramethylhexadecyl)-6,7-dimethoxy-3-methylnaphthalene-1,4-dione;
2-(3-hydroxy-3,7-dimethyloctyl)-6,7-dimethoxy-3-methylnaphthalene-1,4-dione;
2-(3-hydroxy-3,7-dimethyloct-6-en-1-yl)-6,7-dimethoxy-3-methylnaphthalene-1,4-dione;
2-(3-hydroxy-3,7-dimethyloct-6-en-1-yl)-3,6,7-trimethylnaphthalene-1,4-dione;
2-(3-hydroxy-3,7-dimethyloct-6-en-1-yl)-6,7-dimethylnaphthalene-1,4-dione;
2-(3-hydroxy-3,7-dimethyloct-6-en-1-yl)naphthalene-1,4-dione;
2-(3-hydroxy-3,7-dimethyloctyl)-6,7-dimethoxy-3-methylnaphthalene-1,4-dione;
2-(3-hydroxy-3,7-dimethyloctyl)-6,7-dimethylnaphthalene-1,4-dione;
2-(3-hydroxy-3,7-dimethyloctyl)-3,6,7-trimethylnaphthalene-1,4-dione; and
2-(3-hydroxy-3,7-dimethyloctyl)naphthalene-1,4-dione;
   or any stereoisomer, mixture of stereoisomers, prodrug, metabolite, salt, crystalline form, non-crystalline form, hydrate or solvate thereof.

In another embodiment, the invention embraces a method of treating a mitochondrial disorder, modulating one or more energy biomarkers, normalizing one or more energy biomarkers, or enhancing one or more energy biomarkers, comprising administering to a subject an effective amount of one or more compounds of Formula Ic:

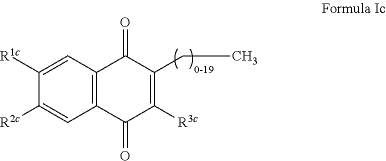

wherein,
R^{1c} and R^{2c} are independently of each other hydrogen; —(C₁-C₆)alkyl or —O(C₁-C₆)alkyl;
R^{1c} is (C₁-C₆)alkyl;
or any prodrug, metabolite, salt, crystalline form, non-crystalline form, hydrate or solvate thereof.

In one embodiment, R^{1c}, R^{2c} and R^{3c} are independently of each other —(C₁-C₆)alkyl. In another embodiment, R^{1c}, R^{2c} and R^{3c} are methyl. In another embodiment, R^{1c} and R^{2c} are hydrogen and R^{3c} is —(C₁-C₆)alkyl. In another embodiment, R^{1c} and R^{2c} are hydrogen and R^{1c} is methyl. In another embodiment, R^{1c} and R^{2c} are hydrogen and R^{3c} is ethyl. In another embodiment, R^{1c} and R^{2c} are hydrogen and R^{3c} is propyl. In another embodiment, R^{1c} and R^{2c} are hydrogen and R^{3c} is butyl. In another embodiment, R^{1c} and R^{2c} are hydrogen and R^{3c} is pentyl. In another embodiment, R^{1c} and R^{2c} are hydrogen and R^{3c} is hexyl. In another embodiment, R^{1c} and R^{2c} are independently of each other —O(C₁-C₆)alkyl and R^{3c} is —(C₁-C₆)alkyl. In another embodiment, R^{1c} and R^{2c} are independently of each other methoxy and R^{3c} is methyl.

In some embodiments, the invention additionally embraces a method of treating a mitochondrial disorder, modulating one or more energy biomarkers, normalizing one or more energy biomarkers, or enhancing one or more energy biomarkers, comprising administering to a subject an effective amount of one or more compounds of Formula I or Formula Ic selected from:
2-ethyl-3-methylnaphthalene-1,4-dione;
2-ethyl-3,6,7-trimethylnaphthalene-1,4-dione;
2-ethyl-6,7-dimethoxy-3-methylnaphthalene-1,4-dione;
2-methyl-3-propylnaphthalene-1,4-dione;
2,6,7-trimethyl-3-propylnaphthalene-1,4-dione;
6,7-dimethoxy-2-methyl-3-propylnaphthalene-1,4-dione;
2-butyl-3-methylnaphthalene-1,4-dione;
2-butyl-3,6,7-trimethylnaphthalene-1,4-dione;
2-butyl-6,7-dimethoxy-3-methylnaphthalene-1,4-dione;
2-methyl-3-pentylnaphthalene-1,4-dione;
2,6,7-trimethyl-3-pentylnaphthalene-1,4-dione;
6,7-dimethoxy-2-methyl-3-pentylnaphthalene-1,4-dione;
2-hexyl-3-methylnaphthalene-1,4-dione;
2-hexyl-3,6,7-trimethylnaphthalene-1,4-dione;
2-hexyl-6,7-dimethoxy-3-methylnaphthalene-1,4-dione;
2-heptyl-3-methylnaphthalene-1,4-dione;
2-heptyl-3,6,7-trimethylnaphthalene-1,4-dione;
2-heptyl-6,7-dimethoxy-3-methylnaphthalene-1,4-dione;
2-methyl-3-octylnaphthalene-1,4-dione;
2,6,7-trimethyl-3-octylnaphthalene-1,4-dione;
6,7-dimethoxy-2-methyl-3-octylnaphthalene-1,4-dione;
2-methyl-3-nonylnaphthalene-1,4-dione;
2,6,7-trimethyl-3-nonylnaphthalene-1,4-dione;
6,7-dimethoxy-2-methyl-3-nonylnaphthalene-1,4-dione;
2-decyl-3-methylnaphthalene-1,4-dione;
2-decyl-3,6,7-trimethylnaphthalene-1,4-dione; and
2-decyl-6,7-dimethoxy-3-methylnaphthalene-1,4-dione.

In another embodiment, the invention embraces a method of treating a mitochondrial disorder, modulating one or more energy biomarkers, normalizing one or more energy biomarkers, or enhancing one or more energy biomarkers, comprising administering to a subject an effective amount of one or more compounds of Formula Id:

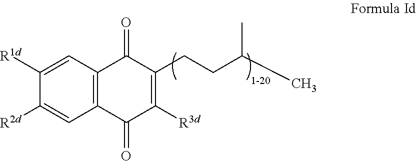

wherein,
R^{1d} and R^{2d} are independently of each other hydrogen; —(C₁-C₆)alkyl or —O(C₁-C₆)alkyl;
R^{3d} is hydrogen or —(C₁-C₆)alkyl;
or any prodrug, metabolite, salt, crystalline form, non-crystalline form, hydrate or solvate thereof.

In one embodiment, R^{1d}, R^{2d} and R^{3d} are independently of each other —(C₁-C₆)alkyl. In another embodiment, R^{1d}, R^{2d} and R^{3d} are methyl. In another embodiment, R^{1d} and R^{2d} are hydrogen and R^{3d} is —(C₁-C₆)alkyl. In another embodiment, R^{1d} and R^{2d} are hydrogen and R^{3d} is methyl. In another embodiment, R^{1d} and R^{2d} are independently of each other —O(C₁-C₆)alkyl and R_{3d} is —(C₁-C₆)alkyl. In another embodiment, R^{1d} and R^{2d} are independently of each other methoxy and R^{3d} is methyl.

In some embodiments, the invention additionally embraces a method of treating a mitochondrial disorder, modulating one or more energy biomarkers, normalizing one or more energy biomarkers, or enhancing one or more energy biomarkers, comprising administering to a subject an effective amount of one or more compounds of Formula I or Formula Id selected from:
2-isopentyl-3-methylnaphthalene-1,4-dione;
2-isopentyl-3,6,7-trimethylnaphthalene-1,4-dione;
2-isopentyl-6,7-dimethoxy-3-methylnaphthalene-1,4-dione;
2-(3,7-dimethyloctyl)-3-methylnaphthalene-1,4-dione;
2-(3,7-dimethyloctyl)-6,7-dimethoxy-3-methylnaphthalene-1,4-dione;
2-(3,7-dimethyloctyl)-3,6,7-trimethylnaphthalene-1,4-dione;
2-methyl-3-(3,7,11-trimethyldodecyl)naphthalene-1,4-dione;
6,7-dimethoxy-2-methyl-3-(3,7,11-trimethyldodecyl)naphthalene-1,4-dione;
2,6,7-trimethyl-3-(3,7,11-trimethyldodecyl)naphthalene-1,4-dione;
2-methyl-3-(3,7,11,15-tetramethylhexadecyl)naphthalene-1,4-dione;
2,6,7-trimethyl-3-(3,7,11,15-tetramethylhexadecyl)naphthalene-1,4-dione;
6,7-dimethoxy-2-methyl-3-(3,7,11,15-tetramethylhexadecyl)naphthalene-1,4-dione;
2,6,7-trimethyl-3-(3,7,11,15,19-pentamethylicosyl)naphthalene-1,4-dione;
6,7-dimethoxy-2-methyl-3-(3,7,11,15,19-pentamethylicosyl)naphthalene-1,4-dione;
2-methyl-3-(3,7,11,15,19-pentamethylicosyl)naphthalene-1,4-dione;
2-(3,7,11,15,19,23-hexamethyltetracosyl)-3-methylnaphthalene-1,4-dione;
2-(3,7,11,15,19,23-hexamethyltetracosyl)-6,7-dimethoxy-3-methylnaphthalene-1,4-dione; and
2-(3,7,11,15,19,23-hexamethyltetracosyl)-3,6,7-trimethylnaphthalene-1,4-dione.

In some embodiments, the invention embraces a method of treating a mitochondrial disorder, modulating one or more energy biomarkers, normalizing one or more energy biomarkers, or enhancing one or more energy biomarkers, comprising administering to a subject an effective amount of one or more compounds of Formula I selected from:
2,3,-dimethylnaphthalene-1,4-dione;
2-butyl-3-methylnaphthalene-1,4-dione;
2-hexyl-3-methylnaphthalene-1,4-dione;
2,3-dimethoxynaphthalene-1,4-dione
2-methoxynaphthalene-1,4-dione; and
naphthalene-1,4-dione;
   or any salt, crystalline form, non-crystalline form, hydrate or solvate thereof.

In any of the methods above, the invention embraces a method of treating, preventing, or suppressing symptoms associated with a mitochondrial disorder, modulating one or more energy biomarkers, normalizing one or more energy biomarkers, or enhancing one or more energy biomarkers, comprising administering to a subject an effective amount of one or more compounds of the Formula I, Formula Ia, Formula Ib, Formula Ic or Formula Id and an acceptable carrier, excipient or vehicle.

In any of the methods above, the mitochondrial disorder can be selected from the group consisting of inherited mitochondrial diseases; Myoclonic Epilepsy with Ragged Red Fibers (MERRF); Mitochondrial Myopathy, Encephalopathy, Lactacidosis, Stroke (MELAS); Leber's Hereditary Optic Neuropathy (LHON); Dominant Optic atrophy (DOA); Leigh syndrome; Kearns-Sayre Syndrome (KSS); Friedreich's Ataxia (FRDA); other myopathies; cardiomyopathy; encephalomyopathy; renal tubular acidosis; Parkinson's disease; Alzheimer's disease; amyotrophic lateral sclerosis (ALS); Huntington's Disease; developmental pervasive disorders or hearing loss.

In another embodiment, the mitochondrial disorder can be selected from the group consisting of inherited mitochondrial diseases; Myoclonic Epilepsy with Ragged Red Fibers (MERRF); Mitochondrial Myopathy, Encephalopathy, Lactacidosis, Stroke (MELAS); Leber's Hereditary Optic Neuropathy (LHON); Dominant Optic atrophy (DOA); Leigh syndrome; Kearns-Sayre Syndrome (KSS); and Friedreich's Ataxia (FRDA).

In any of the methods above, the invention also embraces a method of treating preventing or suppressing the symptoms of diseases resulting from acquired mitochondrial dysfunction, such as neurodegenerative disorders associated with aging like Parkinson's, Alzheimer's and Huntington's disease, disorders associated with cerebral vascular accidents, seizures and ischemia. In some of the methods above the invention also embraces a method of treating, preventing or suppressing the symptoms of autism and developmental pervasive disorders. In some of the methods above the invention also embraces a method of treating, preventing or suppressing hearing disorders such as sensorineural hearing loss.

In any of the methods above for methods of modulating one or more energy biomarkers, normalizing one or more energy biomarkers, or enhancing one or more energy biomarkers, the energy biomarker can be selected from the group consisting of: lactic acid (lactate) levels, either in whole blood, plasma, cerebrospinal fluid, or cerebral ventricular fluid; pyruvic acid (pyruvate) levels, either in whole blood, plasma, cerebrospinal fluid, or cerebral ventricular fluid; lactate/pyruvate ratios, either in whole blood, plasma, cerebrospinal fluid, or cerebral ventricular fluid; phosphocreatine levels, NADH (NADH+H⁺) levels; NADPH (NADPH+H⁺) levels; NAD levels; NADP levels; ATP levels; reduced coenzyme Q (CoQ^{red}) levels; oxidized coenzyme Q (CoQ^{OX}) levels; total coenzyme Q (CoQ^{tot}) levels; oxidized cytochrome C levels; reduced cytochrome C levels; oxidized cytochrome C/reduced cytochrome C ratio; acetoacetate levels, .beta.-hydroxy butyrate levels, acetoacetate/.beta.-hydroxy butyrate ratio, 8-hydroxy-2′-deoxyguanosine (8-OHdG) levels; levels of reactive oxygen species; levels of oxygen consumption (VO2); levels of carbon dioxide output (VCO2); respiratory quotient (VCO2/VO2); exercise tolerance; and anaerobic threshold.

In any of the above methods, the subject can be selected from the group consisting of: a subject with a mitochondrial disease; a subject undergoing strenuous or prolonged physical activity; a subject with chronic energy problems; a subject with chronic respiratory problems; a pregnant female; a pregnant female in labor; a neonate; a premature neonate; a subject exposed to an extreme environment; a subject exposed to a hot environment; a subject exposed to a cold environment; a subject exposed to an environment with lower-than-average oxygen content; a subject exposed to an environment with higher-than-average carbon dioxide content; a subject exposed to an environment with higher-than-average levels of air pollution; a subject with lung disease; a subject with lower-than-average lung capacity; a tubercular patient; a lung cancer patient; an emphysema patient; a cystic fibrosis patient; a subject recovering from surgery; a subject recovering from illness; a subject undergoing acute trauma; a subject in shock; a subject requiring acute oxygen administration; a subject requiring chronic oxygen administration; an elderly subject; an elderly subject experiencing decreased energy; and a subject suffering from chronic fatigue; subjects suffering from chronic fatigue syndrome; subjects undergoing acute trauma; subjects in shock; subjects requiring acute oxygen administration; subjects requiring chronic oxygen administration; or other subjects with acute, chronic, or ongoing energy demands who can benefit from enhancement of energy biomarkers.

In another embodiment, the invention embraces a method of treating, preventing or suppressing a mitochondrial disorder, modulating one or more energy biomarkers, normalizing one or more energy biomarkers, or enhancing one or more energy biomarkers, by administering an effective amount of one or more compounds of Formula I, Formula Ia, Formula Ib, Formula Ic or Formula Id.

In other embodiments, including any of the foregoing embodiments, the mitochondrial disorder is selected from the group consisting of inherited mitochondrial diseases; Myoclonic Epilepsy with Ragged Red Fibers (MERRF); Mitochondrial Myopathy, Encephalopathy, Lactacidosis, Stroke (MELAS); Leber's Hereditary Optic Neuropathy (LHON); dominant optic atrophy (DOA); Leigh syndrome; Kearns-Sayre Syndrome (KSS); Friedreich's Ataxia (FRDA); other myopathies; cardiomyopathy; encephalomyopathy; renal tubular acidosis; neurodegenerative diseases; Parkinson's disease; Alzheimer's disease; amyotrophic lateral sclerosis (ALS); motor neuron diseases; other neurological diseases; epilepsy; genetic diseases; Huntington's Disease; mood disorders; schizophrenia; bipolar disorder; and age-associated diseases.

In another embodiment, including any of the foregoing embodiments, the mitochondrial disorder is selected from the group consisting of inherited mitochondrial diseases; Myoclonic Epilepsy with Ragged Red Fibers (MERRF); Mitochondrial Myopathy, Encephalopathy, Lactacidosis, Stroke (MELAS); Leber's Hereditary Optic Neuropathy (LHON); Dominant Optic atrophy (DOA); Leigh syndrome; Kearns-Sayre Syndrome (KSS); and Friedreich's Ataxia (FRDA).

In another embodiment of the invention, including any of the foregoing embodiments, the mitochondrial disorder is Friedreich's ataxia (FRDA). In another embodiment of the invention, the mitochondrial disorder is Leber's Hereditary Optic Neuropathy (LHON). In another embodiment of the invention, the mitochondrial disorder is Dominant Optic atrophy (DOA). In another embodiment of the invention, the mitochondrial disorder is mitochondrial myopathy, encephalopathy, lactacidosis, stroke (MELAS). In another embodiment of the invention, the mitochondrial disorder is Kearns-Sayre Syndrome (KSS). In another embodiment of the invention, the mitochondrial disorder is Myoclonic Epilepsy with Ragged Red Fibers (MERRF). In another embodiment of the invention, the mitochondrial disorder is Parkinson's disease. In another embodiment of the invention, the mitochondrial disorder is Leigh syndrome. In another embodiment of the invention, the mitochondrial disorder is Leigh syndrome with a SURF1 mutation.

In another embodiment of the invention, the mitochondrial dysfunction contributes to Huntington's disease. In another embodiment of the invention, the mitochondrial dysfunction contributes to amyotrophic lateral sclerosis (ALS). In another embodiment of the invention, the mitochondrial dysfunction contributes to Parkinson's disease. In another embodiment of the invention, the mitochondrial dysfunction contributes to a disorder associated with cerebral vascular accidents, seizures and ischemia. In another embodiment of the invention, the mitochondrial dysfunction contributes to autism or a developmental pervasive disorder. In another embodiment of the invention, the mitochondrial dysfunction contributes to a hearing disorder such as sensorineural hearing loss.

In another embodiment of the invention, including any of the foregoing embodiments, the compounds described herein are administered to subjects suffering from a mitochondrial disorder or dysfunction to modulate one or more of various energy biomarkers, including, but not limited to, lactic acid (lactate) levels, either in whole blood, plasma, cerebrospinal fluid, or cerebral ventricular fluid; pyruvic acid (pyruvate) levels, either in whole blood, plasma, cerebrospinal fluid, or cerebral ventricular fluid; lactate/pyruvate ratios, either in whole blood, plasma, cerebrospinal fluid, or cerebral ventricular fluid; phosphocreatine levels, NADH (NADH+H⁺) or NADPH (NADPH+H⁺) levels; NAD or NADP levels; ATP levels; reduced coenzyme Q (CoQ^{red}) levels; oxidized coenzyme Q (CoQ^{OX}) levels; total coenzyme Q (CoQ^{tot}) levels; oxidized cytochrome C levels; reduced cytochrome C levels; oxidized cytochrome C/reduced cytochrome C ratio; acetoacetate levels; beta-hydroxy butyrate levels; acetoacetate/beta-hydroxy butyrate ratio; 8-hydroxy-2′-deoxyguanosine (8-OHdG) levels; levels of reactive oxygen species; oxygen consumption (VO2), carbon dioxide output (VCO2), respiratory quotient (VCO2/VO2), and to modulate exercise intolerance (or conversely, modulate exercise tolerance) and to modulate anaerobic threshold. Energy biomarkers can be measured in whole blood, plasma, cerebrospinal fluid, cerebroventricular fluid, arterial blood, venous blood, or any other body fluid, body gas, or other biological sample useful for such measurement. In one embodiment, the levels are modulated to a value within about 2 standard deviations of the value in a healthy subject. In another embodiment, the levels are modulated to a value within about 1 standard deviation of the value in a healthy subject. In another embodiment, the levels in a subject are changed by at least about 10% above or below the level in the subject prior to modulation. In another embodiment, the levels are changed by at least about 20% above or below the level in the subject prior to modulation. In another embodiment, the levels are changed by at least about 30% above or below the level in the subject prior to modulation. In another embodiment, the levels are changed by at least about 40% above or below the level in the subject prior to modulation. In another embodiment, the levels are changed by at least about 50% above or below the level in the subject prior to modulation. In another embodiment, the levels are changed by at least about 75% above or below the level in the subject prior to modulation. In another embodiment, the levels are changed by at least about 100% above or at least about 90% below the level in the subject prior to modulation.

In another embodiment, including any of the foregoing embodiments, the subject or subjects in which a method of treating or suppressing a mitochondrial disorder, modulating one or more energy biomarkers, normalizing one or more energy biomarkers, or enhancing one or more energy biomarkers is performed is/are selected from the group consisting of subjects undergoing strenuous or prolonged physical activity; subjects with chronic energy problems; subjects with chronic respiratory problems; pregnant females; pregnant females in labor; neonates; premature neonates; subjects exposed to extreme environments; subjects exposed to hot environments; subjects exposed to cold environments; subjects exposed to environments with lower-than-average oxygen content; subjects exposed to environments with higher-than-average carbon dioxide content; subjects exposed to environments with higher-than-average levels of air pollution; airline travelers; flight attendants; subjects at elevated altitudes; subjects living in cities with lower-than-average air quality; subjects working in enclosed environments where air quality is degraded; subjects with lung diseases; subjects with lower-than-average lung capacity; tubercular patients; lung cancer patients; emphysema patients; cystic fibrosis patients; subjects recovering from surgery; subjects recovering from illness; elderly subjects; elderly subjects experiencing decreased energy; subjects suffering from chronic fatigue; subjects suffering from chronic fatigue syndrome; subjects undergoing acute trauma; subjects in shock; subjects requiring acute oxygen administration; subjects requiring chronic oxygen administration; or other subjects with acute, chronic, or ongoing energy demands who can benefit from enhancement of energy biomarkers.

In another embodiment, including any of the foregoing embodiments, the invention embraces one or more compounds described herein in combination with a nutritionally acceptable excipient, carrier, or vehicle. In another embodiment, including any of the foregoing embodiments, the invention embraces one or more compounds described herein in combination with a therapeutically acceptable excipient, carrier, or vehicle

In another embodiment, the invention embraces the use of one or more compounds described herein in therapy. In another embodiment, the invention embraces the use of one or more compounds described herein in the treatment, prevention or suppression of symptoms associated with mitochondrial disease or dysfunction. In another embodiment, the invention embraces the use of one or more compounds described herein in the manufacture of a medicament for use in treatment, prevention or suppression of symptoms associated with a mitochondrial disease or dysfunction.

For all of the compounds and methods described above, the naphthoquinone form can also be used in its reduced (naphthoquinol) form when desired.

### MODES FOR CARRYING OUT THE INVENTION

The invention embraces compounds useful in treating or suppressing mitochondrial disorders, and methods of using such compounds for modulation of energy biomarkers. The redox active therapeutics for treatment or suppression of mitochondrial diseases and associated aspects of the invention are described in more detail herein.

By “subject,” “individual,” or “patient” is meant an individual organism, preferably a vertebrate, more preferably a mammal, most preferably a human.

“Treating” a disease with the compounds and methods discussed herein is defined as administering one or more of the compounds discussed herein, with or without additional therapeutic agents, in order to reduce or eliminate either the disease or one or more symptoms of the disease, or to retard the progression of the disease or of one or more symptoms of the disease, or to reduce the severity of the disease or of one or more symptoms of the disease. “Suppression” of a disease with the compounds and methods discussed herein is defined as administering one or more of the compounds discussed herein, with or without additional therapeutic agents, in order to suppress the clinical manifestation of the disease, or to suppress the manifestation of adverse symptoms of the disease. The distinction between treatment and suppression is that treatment occurs after adverse symptoms of the disease are manifest in a subject, while suppression occurs before adverse symptoms of the disease are manifest in a subject. Suppression may be partial, substantially total, or total. Because many of the mitochondrial disorders are inherited, genetic screening can be used to identify patients at risk of the disease. The compounds and methods of the invention can then be administered to asymptomatic patients at risk of developing the clinical symptoms of the disease, in order to suppress the appearance of any adverse symptoms. “Therapeutic use” of the compounds discussed herein is defined as using one or more of the compounds discussed herein to treat or suppress a disease, as defined above. An “effective amount” of a compound is an amount of a compound which, when administered to a subject, is sufficient to reduce or eliminate either one or more symptoms of a disease, or to retard the progression of one or more symptoms of a disease, or to reduce the severity of one or more symptoms of a disease, or to suppress the manifestation of a disease, or to suppress the manifestation of adverse symptoms of a disease. An effective amount can be given in one or more administrations. An “effective amount” of a compound embraces both a therapeutically effective amount, as well as an amount effective to modulate, normalize, or enhance one or more energy biomarkers in a subject.

“Modulation” of, or to “modulate,” an energy biomarker means to change the level of the energy biomarker towards a desired value, or to change the level of the energy biomarker in a desired direction (e.g., increase or decrease). Modulation can include, but is not limited to, normalization and enhancement as defined below.

“Normalization” of, or to “normalize,” an energy biomarker is defined as changing the level of the energy biomarker from a pathological value towards a normal value, where the normal value of the energy biomarker can be 1) the level of the energy biomarker in a healthy person or subject, or 2) a level of the energy biomarker that alleviates one or more undesirable symptoms in the person or subject. That is, to normalize an energy biomarker which is depressed in a disease state means to increase the level of the energy biomarker towards the normal (healthy) value or towards a value which alleviates an undesirable symptom; to normalize an energy biomarker which is elevated in a disease state means to decrease the level of the energy biomarker towards the normal (healthy) value or towards a value which alleviates an undesirable symptom.

“Enhancement” of, or to “enhance,” energy biomarkers means to intentionally change the level of one or more energy biomarkers away from either the normal value, or the value before enhancement, in order to achieve a beneficial or desired effect. For example, in a situation where significant energy demands are placed on a subject, it may be desirable to increase the level of ATP in that subject to a level above the normal level of ATP in that subject. Enhancement can also be of beneficial effect in a subject suffering from a disease or pathology such as a mitochondrial disease, in that normalizing an energy biomarker may not achieve the optimum outcome for the subject; in such cases, enhancement of one or more energy biomarkers can be beneficial, for example, higher-than-normal levels of ATP, or lower-than-normal levels of lactic acid (lactate) can be beneficial to such a subject.

By modulating, normalizing, or enhancing the energy biomarker Coenzyme Q is meant modulating, normalizing, or enhancing the variant or variants of Coenzyme Q which is predominant in the species of interest. For example, the variant of Coenzyme Q which predominates in humans is Coenzyme Q10. If a species or subject has more than one variant of Coenzyme Q present in significant amounts (i.e., present in amounts which, when modulated, normalized, or enhanced, can have a beneficial effect on the species or subject), modulating, normalizing, or enhancing Coenzyme Q can refer to modulating, normalizing or enhancing any or all variants of Coenzyme Q present in the species or subject.

While the compounds described herein can occur and can be used as the neutral (non-salt) compound, the description is intended to embrace all salts of the compounds described herein, as well as methods of using such salts of the compounds. In one embodiment, the salts of the compounds comprise pharmaceutically acceptable salts. Pharmaceutically acceptable salts are those salts which can be administered as drugs or pharmaceuticals to humans and/or animals and which, upon administration, retain at least some of the biological activity of the free compound (neutral compound or non-salt compound). The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, can also be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminum salts. Examples of organic salts of acid compounds include, but are not limited to, procaine, dibenzylamine, N-ethylpiperidine, N,N′-dibenzylethylenediamine, and triethylamine salts. Salts of acidic compounds with amino acids, such as lysine salts, can also be prepared.

The invention also includes all stereoisomers and geometric isomers of the compounds, including diastereomers, enantiomers, and cis/trans (E/Z) isomers. The invention also includes mixtures of stereoisomers and/or geometric isomers in any ratio, including, but not limited to, racemic mixtures.

The compounds can be administered in prodrug form. Prodrugs are derivatives of the compounds which are themselves relatively inactive, but which convert into the active compound when introduced into the subject in which they are used, by a chemical or biological process in vivo, such as an enzymatic conversion. Suitable prodrug formulations include, but are not limited to, peptide conjugates of the compounds of the invention and esters of compounds of the inventions. Further discussion of suitable prodrugs is provided in H. Bundgaard, Design of Prodrugs, New York: Elsevier, 1985; in R. Silverman, The Organic Chemistry of Drug Design and Drug Action, Boston: Elsevier, 2004; in R. L. Juliano (ed.), Biological Approaches to the Controlled Delivery of Drugs (Annals of the New York Academy of Sciences, v. 507), New York: N.Y. Academy of Sciences, 1987; and in E. B. Roche (ed.), Design of Biopharmaceutical Properties Through Prodrugs and Analogs (Symposium sponsored by Medicinal Chemistry Section, APhA Academy of Pharmaceutical Sciences, November 1976 national meeting, Orlando, Fla.), Washington: The Academy, 1977.

The various compounds of the invention can be administered either as therapeutic agents in and of themselves, or as prodrugs which will convert to other therapeutically effective or effective substances in the body.

The term “alkyl” refers to saturated aliphatic groups including straight-chain, branched-chain, cyclic groups, and combinations thereof, having the number of carbon atoms specified, or if no number is specified, having up to 12 carbon atoms. “Straight-chain alkyl” or “linear alkyl” group refers to alkyl groups that are neither cyclic nor branched, commonly designated as “n-alkyl” groups. One subset of alkyl groups is —(C₁-C₆)alkyl which include groups such as methyl, ethyl, n-propyl, isopropyl, butyl, n-butyl, isobutyl, sec-butyl, t-butyl, pentyl, n-pentyl, hexyl, cyclopropyl, cyclobutyl, cyclopentyl, cyclohexyl, and any other alkyl group containing between one and five carbon atoms, where the —(C₁-C₆)alkyl groups can be attached via any valence on the —(C₁-C₆) alkyl groups.

Some compounds of interest, which can be used in any of the methods of the invention are:
2-(3-hydroxy-3,7,11,15-tetramethylhexadeca-6,10,14-trien-1-yl)-3-methylnaphthalene-1,4-dione (CAS Registry Number 159489-25-5)
2,3,6-trimethylnaphthalene-1,4-dione (CAS Registry Number 20490-42-0);
2-ethyl-3-methylnaphthalene-1,4-dione (CAS Registry Number 2589-56-2);
2-propyl-3-methylnaphthalene-1,4-dione (CAS Registry Number 2397-61-7);
2-butyl-3-methylnaphthalene-1,4-dione (CAS Registry Number 2397-62-8);
2-hexyl-3-methylnaphthalene-1,4-dione (CAS Registry Number 170967-35-8);
2-nonyl-3-methylnaphthalene-1,4-dione (CAS Registry Number 134650-31-0);
2-decyl-3-methylnaphthalene-1,4-dione (CAS Registry Number 117157-41-2);
2,6-dimethyl-3-undecylnaphthalene-1,4-dione (CAS Registry Number 245072-32-6);
2,6,7-trimethyl-3-undecylnaphthalene-1,4-dione (CAS Registry Number 245072-36-0);
3,6-dimethyl-2-undecylnaphthalene-1,4-dione (CAS Registry Number 245072-33-7);
2-methyl-3-tridecylnaphthalene-1,4-dione (CAS Registry Number 134650-33-2);
2-methyl-3-tetradecylnaphthalene-1,4-dione (CAS Registry Number 848401-03-6);
2-methyl-3-pentadecylnaphthalene-1,4-dione (CAS Registry Number 70691-74-6)
2-methyl-3-heptadecylnaphthalene-1,4-dione (CAS Registry Number 96378-21-1);
2-methyl-3-nonadecylnaphthalene-1,4-dione (CAS Registry Number 848401-04-7);
2-icosyl-3-methylnaphthalene-1,4-dione (CAS Registry Number 118709-75-4);
2-methyl-3-(3,7,11,15-tetramethylhexadecyl)naphthalene-1,4-dione (CAS Registry Number 1217521-73-7);
2,6-dimethyl-3-(3,7,11,15-tetramethylhexadecyl)naphthalene-1,4-dione (CAS Registry Number 8602471-23-0);
2-methyl-3-(3,7,11,15,19-pentamethylicosyl)naphthalene-1,4-dione (CAS Registry Number 100364-50-9);
2-(3,7,11,15,19,23-hexamethyltetracosyl)-3-methylnaphthalene-1,4-dione (CAS Registry Number 100364-52-1);
2-(3,7,11,15,19,23,27-heptamethyloctacosyl)-3-methylnaphthalene-1,4-dione (CAS Registry Number 120551-48-6);
2-methyl-3-(3,7,11,15,19,23,27,31,35-nonamethylhexatriacontyl)naphthalene-1,4-dione (CAS Registry Number 47897-73-4);
2-(3,7,11,15,19,23,27,31,35,39-decamethyltetracontyl)-3-methylnaphthalene-1,4-dione (CAS Registry Number 47986-59-2);
2,3,-dimethylnaphthalene-1,4-dione (CAS Registry Number 2195-57-1);
2-butyl-3-methylnaphthalene-1,4-dione (CAS Registry Number 2397-62-8);
2-hexyl-3-methylnaphthalene-1,4-dione (CAS Registry Number 170967-35-8);
2,3-dimethoxynaphthalene-1,4-dione (CAS Registry Number 6956-96-3);
2-methoxynaphthalene-1,4-dione (CAS Registry Number 2348-82-5);
naphthalene-1,4-dione (CAS Registry Number 130-15-4);
6,7-dimethoxy-2-methyl-3-(3,7,11,15-tetramethylhexadecyl)naphthalene-1,4-dione (CAS Registry Number 107926-85-2); and
6,7-dimethoxy-2-methyl-3-(3,7,11,15-tetramethylhexadeca-2,6,10,14-tetraen-1-yl)naphthalene-1,4-dione (CAS Registry Number 105403-24-5);
   or any stereoisomer, mixture of stereoisomers, salt, crystalline form, non-crystalline form, hydrate or solvate thereof.

### Synthesis and Sources of Compounds

The compounds of the present invention can be readily synthesized by a variety of methods known in the art. The syntheses of some of the compounds described herein are detailed in, for example, in Isler, O.; et al., Helvetica Chimica Acta (1958), 41, 786-807 or Isler, O. et al. Chimia (1958), 12, 69. Preparation processes of naphthoquinone derivatives of the invention have also been covered in the patent literature, for example in U.S. Pat. Nos. 4,374,775; 4,906,411; 5,329,026; 5,412,124; 5,476,955; 5,637,741, 5,677,471; 5,770,774; 6,579,994; and in PCT No. WO/2008/031283. Additional syntheses of Vitamin K analogues have been described in Weichet J. et al., Collection of Czechoslovak Chemical Communications (1964) 29, 197-205.

### In Vitro Assessment of Efficacy of Compounds

The compounds of the invention can be tested in vitro for efficacy. One such assay is ability of a compound to rescue FRDA fibroblasts stressed by addition of L-buthionine-(S,R)-sulfoximine (BSO), as described in Jauslin et al., Hum. Mol. Genet. 11(24):3055 (2002), Jauslin et al., FASEB J. 17:1972-4 (2003), and International Patent Application WO 2004/003565. Human dermal fibroblasts from Friedreich's ataxia patients have been shown to be hypersensitive to inhibition of the de novo synthesis of glutathione (GSH) with L-buthionine-(S,R)-sulfoximine (BSO), a specific inhibitor of GSH synthetase (Jauslin et al., Hum. Mol. Genet. 11(24):3055 (2002)). This specific BSO-mediated cell death can be prevented by administration of antioxidants or molecules involved in the antioxidant pathway, such as .alpha.-tocopherol, short chain quinones, selenium, or small molecule glutathione peroxidase mimetics. However, antioxidants differ in their potency, i.e. the concentration at which they are able to rescue BSO-stressed FRDA fibroblasts. With this assay, EC₅₀ concentrations of the compounds of the invention can be determined and compared to known reference antioxidants. Similarly the compound of the invention can be tested in vitro for efficacy with assays using fibroblasts from cells from patients with other diseases caused by mitochondrial mutations, such as LHON; Leigh syndrome; SURF1; Huntington's; Parkinson's; MELAS; MERFF; and CoQ10 deficiency.

### Clinical Assessment of Mitochondrial Dysfunction and Efficacy of Therapy

Several readily measurable clinical markers are used to assess the metabolic state of patients with mitochondrial disorders. These markers can also be used as indicators of the efficacy of a given therapy, as the level of a marker is moved from the pathological value to the healthy value. These clinical markers include, but are not limited to, one or more of the previously discussed energy biomarkers, such as lactic acid (lactate) levels, either in whole blood, plasma, cerebrospinal fluid, or cerebral ventricular fluid; pyruvic acid (pyruvate) levels, either in whole blood, plasma, cerebrospinal fluid, or cerebral ventricular fluid; lactate/pyruvate ratios, either in whole blood, plasma, cerebrospinal fluid, or cerebral ventricular fluid; phosphocreatine levels, NADH (NADH+H⁺) or NADPH (NADPH+H⁺) levels; NAD or NADP levels; ATP levels; anaerobic threshold; reduced coenzyme Q (CoQ^{red}) levels; oxidized coenzyme Q (CoQ^{OX}) levels; total coenzyme Q (CoQ^{tot}) levels; oxidized cytochrome C levels; reduced cytochrome C levels; oxidized cytochrome C/reduced cytochrome C ratio; acetoacetate levels, .beta.-hydroxy butyrate levels, acetoacetate/beta-hydroxy butyrate ratio, 8-hydroxy-2′-deoxyguanosine (8-OHdG) levels; levels of reactive oxygen species; and levels of oxygen consumption (VO2), levels of carbon dioxide output (VCO2), and respiratory quotient (VCO2/VO2). Several of these clinical markers are measured routinely in exercise physiology laboratories, and provide convenient assessments of the metabolic state of a subject. In one embodiment of the invention, the level of one or more energy biomarkers in a patient suffering from a mitochondrial disease, such as Friedreich's ataxia, Leber's hereditary optic neuropathy, dominant optic atrophy, Leigh syndrome, SURF1, MERRF, MELAS, or KSS, is improved to within two standard deviations of the average level in a healthy subject. In another embodiment of the invention, the level of one or more of these energy biomarkers in a patient suffering from a mitochondrial disease, such as Friedreich's ataxia, Leber's hereditary optic neuropathy, dominant optic atrophy, Leigh syndrome, SURF1, MERRF, MELAS, or KSS is improved to within one standard deviation of the average level in a healthy subject. Exercise intolerance can also be used as an indicator of the efficacy of a given therapy, where an improvement in exercise tolerance (i.e., a decrease in exercise intolerance) indicates efficacy of a given therapy.

Several metabolic biomarkers have already been used to evaluate efficacy of CoQ10, and these metabolic biomarkers can be monitored as energy biomarkers for use in the methods of the current invention. Pyruvate, a product of the anaerobic metabolism of glucose, is removed by reduction to lactic acid in an anaerobic setting or by oxidative metabolism, which is dependent on a functional mitochondrial respiratory chain. Dysfunction of the respiratory chain may lead to inadequate removal of lactate and pyruvate from the circulation and elevated lactate/pyruvate ratios are observed in mitochondrial cytopathies (see Scriver C R, The metabolic and molecular bases of inherited disease, 7th ed., New York: McGraw-Hill, Health Professions Division, 1995; and Munnich et al., J. Inherit. Metab. Dis. 15(4):448-55 (1992)). Blood lactate/pyruvate ratio (Chariot et al., Arch. Pathol. Lab. Med. 118(7):695-7 (1994)) is, therefore, widely used as a noninvasive test for detection of mitochondrial cytopathies (see again Scriver C R, The metabolic and molecular bases of inherited disease, 7th ed., New York: McGraw-Hill, Health Professions Division, 1995; and Munnich et al., J. Inherit. Metab. Dis. 15(4):448-55 (1992)) and toxic mitochondrial myopathies (Chariot et al., Arthritis Rheum. 37(4):583-6 (1994)). Changes in the redox state of liver mitochondria can be investigated by measuring the arterial ketone body ratio (acetoacetate/3-hydroxybutyrate:AKBR) (Ueda et al., J. Cardiol. 29(2):95-102 (1997)). Urinary excretion of 8-hydroxy-2′-deoxyguanosine (8-OHdG) often has been used as a biomarker to assess the extent of repair of ROS-induced DNA damage in both clinical and occupational settings (Erhola et al., FEBS Lett. 409(2):287-91 (1997); Honda et al., Leuk. Res. 24(6):461-8 (2000); Pilger et al., Free Radic. Res. 35(3):273-80 (2000); Kim et al. Environ Health Perspect 112(6):666-71 (2004)).

Magnetic resonance spectroscopy (MRS) has been useful in the diagnoses of mitochondrial cytopathy by demonstrating elevations in cerebrospinal fluid (CSF) and cortical white matter lactate using proton MRS (¹H-MRS) (Kaufmann et al., Neurology 62(8):1297-302 (2004)). Phosphorous MRS (31P-MRS) has been used to demonstrate low levels of cortical phosphocreatine (PCr) (Matthews et al., Ann. Neurol. 29(4):435-8 (1991)), and a delay in PCr recovery kinetics following exercise in skeletal muscle (Matthews et al., Ann. Neurol. 29(4):435-8 (1991); Barbiroli et al., J. Neurol. 242(7):472-7 (1995); Fabrizi et al., J. Neurol. Sci. 137(1):20-7 (1996)). A low skeletal muscle PCr has also been confirmed in patients with mitochondrial cytopathy by direct biochemical measurements.

Exercise testing is particularly helpful as an evaluation and screening tool in mitochondrial myopathies. One of the hallmark characteristics of mitochondrial myopathies is a reduction in maximal whole body oxygen consumption (VO2max) (Taivassalo et al., Brain 126(Pt 2):413-23 (2003)). Given that VO2max is determined by cardiac output (Qc) and peripheral oxygen extraction (arterial-venous total oxygen content) difference, some mitochondrial cytopathies affect cardiac function where delivery can be altered; however, most mitochondrial myopathies show a characteristic deficit in peripheral oxygen extraction (A-V O2 difference) and an enhanced oxygen delivery (hyperkinetic circulation) (Taivassalo et al., Brain 126(Pt 2):413-23 (2003)). This can be demonstrated by a lack of exercise induced deoxygenation of venous blood with direct AV balance measurements (Taivassalo et al., Ann. Neurol. 51(1):38-44 (2002)) and non-invasively by near infrared spectroscopy (Lynch et al., Muscle Nerve 25(5):664-73 (2002); van Beekvelt et al., Ann. Neurol. 46(4):667-70 (1999)).

Several of these energy biomarkers are discussed in more detail as follows. It should be emphasized that, while certain energy biomarkers are discussed and enumerated herein, the invention is not limited to modulation, normalization or enhancement of only these enumerated energy biomarkers.

Lactic acid (lactate) levels: Mitochondrial dysfunction typically results in abnormal levels of lactic acid, as pyruvate levels increase and pyruvate is converted to lactate to maintain capacity for glycolysis. Mitochondrial dysfunction can also result in abnormal levels of NADH+H⁺, NADPH+H⁺, NAD, or NADP, as the reduced nicotinamide adenine dinucleotides are not efficiently processed by the respiratory chain. Lactate levels can be measured by taking samples of appropriate bodily fluids such as whole blood, plasma, or cerebrospinal fluid. Using magnetic resonance, lactate levels can be measured in virtually any volume of the body desired, such as the brain.

Measurement of cerebral lactic acidosis using magnetic resonance in MELAS patients is described in Kaufmann et al., Neurology 62(8):1297 (2004). Values of the levels of lactic acid in the lateral ventricles of the brain are presented for two mutations resulting in MELAS, A3243G and A8344G. Whole blood, plasma, and cerebrospinal fluid lactate levels can be measured by commercially available equipment such as the YSI 2300 STAT Plus Glucose & Lactate Analyzer (YSI Life Sciences, Ohio).

NAD, NADP, NADH and NADPH levels: Measurement of NAD, NADP, NADH (NADH+H⁺) or NADPH (NADPH+H⁺) can be measured by a variety of fluorescent, enzymatic, or electrochemical techniques, e.g., the electrochemical assay described in US 2005/0067303.

Oxygen consumption (vO₂ or VO2), carbon dioxide output (vCO₂ or VCO2), and respiratory quotient (VCO2/VO2): vO₂ is usually measured either while resting (resting vO.sub.2) or at maximal exercise intensity (vO₂ max). Optimally, both values will be measured. However, for severely disabled patients, measurement of vO₂ max may be impractical. Measurement of both forms of vO₂ is readily accomplished using standard equipment from a variety of vendors, e.g. Korr Medical Technologies, Inc. (Salt Lake City, Utah). VCO2 can also be readily measured, and the ratio of VCO2 to VO2 under the same conditions (VCO2/VO2, either resting or at maximal exercise intensity) provides the respiratory quotient (RQ).

Oxidized Cytochrome C, reduced Cytochrome C, and ratio of oxidized Cytochrome C to reduced Cytochrome C:Cytochrome C parameters, such as oxidized cytochrome C levels (Cyt C^{ox}), reduced cytochrome C levels (Cyt C^{red}), and the ratio of oxidized cytochrome C/reduced cytochrome C ratio (Cyt C^{ox})/(Cyt C^{red}), can be measured by in vivo near infrared spectroscopy. See, e.g., Rolfe, P., “In vivo near-infrared spectroscopy,” Annu. Rev. Biomed. Eng. 2:715-54 (2000) and Strangman et al., “Non-invasive neuroimaging using near-infrared light” Biol. Psychiatry 52:679-93 (2002).

Exercise tolerance/Exercise intolerance: Exercise intolerance is defined as “the reduced ability to perform activities that involve dynamic movement of large skeletal muscles because of symptoms of dyspnea or fatigue” (Pina et al., Circulation 107:1210 (2003)). Exercise intolerance is often accompanied by myoglobinuria, due to breakdown of muscle tissue and subsequent excretion of muscle myoglobin in the urine. Various measures of exercise intolerance can be used, such as time spent walking or running on a treadmill before exhaustion, time spent on an exercise bicycle (stationary bicycle) before exhaustion, and the like. Treatment with the compounds or methods of the invention can result in about a 10% or greater improvement in exercise tolerance (for example, about a 10% or greater increase in time to exhaustion, e.g. from 10 minutes to 11 minutes), about a 20% or greater improvement in exercise tolerance, about a 30% or greater improvement in exercise tolerance, about a 40% or greater improvement in exercise tolerance, about a 50% or greater improvement in exercise tolerance, about a 75% or greater improvement in exercise tolerance, or about a 100% or greater improvement in exercise tolerance. While exercise tolerance is not, strictly speaking, an energy biomarker, for the purposes of the invention, modulation, normalization, or enhancement of energy biomarkers includes modulation, normalization, or enhancement of exercise tolerance.

Similarly, tests for normal and abnormal values of pyruvic acid (pyruvate) levels, lactate/pyruvate ratio, ATP levels, anaerobic threshold, reduced coenzyme Q (CoQ^{red}) levels, oxidized coenzyme Q (CoQ^{ox}) levels, total coenzyme Q (CoQ^{tot}) levels, oxidized cytochrome C levels, reduced cytochrome C levels, oxidized cytochrome C/reduced cytochrome C ratio, acetoacetate levels, .beta.-hydroxy butyrate levels, acetoacetate/.beta.-hydroxy butyrate ratio, 8-hydroxy-2′-deoxyguanosine (8-OHdG) levels, and levels of reactive oxygen species are known in the art and can be used to evaluate efficacy of the compounds and methods of the invention. (For the purposes of the invention, modulation, normalization, or enhancement of energy biomarkers includes modulation, normalization, or enhancement of anaerobic threshold.)

Table 1, following, illustrates the effect that various dysfunctions can have on biochemistry and energy biomarkers. It also indicates the physical effect (such as a disease symptom or other effect of the dysfunction) typically associated with a given dysfunction. It should be noted that any of the energy biomarkers listed in the table, in addition to energy biomarkers enumerated elsewhere, can also be modulated, enhanced, or normalized by the compounds and methods of the invention. RQ=respiratory quotient; BMR=basal metabolic rate; HR(CO)=heart rate (cardiac output); T=body temperature (preferably measured as core temperature); AT=anaerobic threshold; pH=blood pH (venous and/or arterial).

TABLE 1

| Site of | | Measurable Energy | |
|---|---|---|---|
| Dysfunction | Biochemical Event | Biomarker | Physical Effect |
| Respiratory | ↑ NADH | Δ lactate, | Metabolic |
| Chain | | Δ lactate: pyruvate ratio; | dyscrasia & |
| | | and | fatigue |
| | | Δ acetoacetate: β-hydroxy | |
| | | butyrate ratio | |
| Respiratory | ↓ H⁺ gradient | Δ ATP | Organ dependent |
| Chain | | | dysfunction |
| Respiratory | ↓ Electron flux | Δ VO₂, RQ, BMR, ΔT, | Metabolic |
| Chain | | AT, pH | dyscrasia & |
| | | | fatigue |
| Mitochondria & | ↓ ATP, ↓ VO₂ | Δ Work, ΔHR (CO) | Exercise |
| cytosol | | | intolerance |
| Mitochondria & | ↓ ATP | Δ PCr | Exercise |
| cytosol | | | intolerance |
| Respiratory | ↓ Cyt C_{Ox/Red} | Δ λ ~700-900 nM (Near | Exercise |
| Chain | | Infrared Spectroscopy) | intolerance |
| Intermediary | ↓ Catabolism | Δ C¹⁴-Labeled substrates | Metabolic |
| metabolism | | | dyscrasia & |
| | | | fatigue |
| Respiratory | ↓ Electron flux | Δ Mixed Venous VO₂ | Metabolic |
| Chain | | | dyscrasia & |
| | | | fatigue |
| Mitochondria & | ↑ Oxidative stress | Δ Tocopherol & | Uncertain |
| cytosol | | Tocotrienols, CoQ10, | |
| | | docosahexanoic acid | |
| Mitochondria & | ↑ Oxidative stress | Δ Glutathione_{red} | Uncertain |
| cytosol | | | |
| Mitochondria & | Nucleic acid oxidation | Δ 8-hydroxy 2-deoxy | Uncertain |
| cytosol | | guanosine | |
| Mitochondria & | Lipid oxidation | Δ Isoprostane(s), | Uncertain |
| cytosol | | eicasanoids | |
| Cell membranes | Lipid oxidation | Δ Ethane (breath) | Uncertain |
| Cell membranes | Lipid oxidation | Δ Malondialdehyde | Uncertain |

Treatment of a subject afflicted by a mitochondrial disease in accordance with the methods of the invention may result in the inducement of a reduction or alleviation of symptoms in the subject, e.g., to halt the further progression of the disorder.

Partial or complete suppression of the mitochondrial disease can result in a lessening of the severity of one or more of the symptoms that the subject would otherwise experience. For example, partial suppression of MELAS could result in reduction in the number of stroke-like or seizure episodes suffered.

Any one energy biomarker or any combination of the energy biomarkers described herein provides conveniently measurable benchmarks by which to gauge the effectiveness of treatment or suppressive therapy. Additionally, other energy biomarkers are known to those skilled in the art and can be monitored to evaluate the efficacy of treatment or suppressive therapy.

### Use of Compounds for Modulation of Energy Biomarkers

In addition to monitoring energy biomarkers to assess the status of treatment or suppression of mitochondrial diseases, the compounds of the invention can be used in subjects or patients to modulate one or more energy biomarkers. Modulation of energy biomarkers can be done to normalize energy biomarkers in a subject, or to enhance energy biomarkers in a subject.

Normalization of one or more energy biomarkers is defined as either restoring the level of one or more such energy biomarkers to normal or near-normal levels in a subject whose levels of one or more energy biomarkers show pathological differences from normal levels (i.e., levels in a healthy subject), or to change the levels of one or more energy biomarkers to alleviate pathological symptoms in a subject. Depending on the nature of the energy biomarker, such levels may show measured values either above or below a normal value. For example, a pathological lactate level is typically higher than the lactate level in a normal (i.e., healthy) person, and a decrease in the level may be desirable. A pathological ATP level is typically lower than the ATP level in a normal (i.e., healthy) person, and an increase in the level of ATP may be desirable. Accordingly, normalization of energy biomarkers can involve restoring the level of energy biomarkers to within about at least two standard deviations of normal in a subject, more preferably to within about at least one standard deviation of normal in a subject, to within about at least one-half standard deviation of normal, or to within about at least one-quarter standard deviation of normal.

When an increase in an energy biomarker level is desired to normalize the one or more such energy biomarker, the level of the energy biomarker can be increased to within about at least two standard deviations of normal in a subject, more preferably increased to within about at least one standard deviation of normal in a subject, increased to within about at least one-half standard deviation of normal, or increased to within about at least one-quarter standard deviation of normal, by administration of one or more compounds according to the invention. Alternatively, the level of one or more of the energy biomarkers can be increased by about at least 10% above the subject's level of the respective one or more energy biomarkers before administration; by about at least 20% above the subject's level of the respective one or more energy biomarkers before administration, by about at least 30% above the subject's level of the respective one or more energy biomarkers before administration, by about at least 40% above the subject's level of the respective one or more energy biomarkers before administration, by about at least 50% above the subject's level of the respective one or more energy biomarkers before administration, by about at least 75% above the subject's level of the respective one or more energy biomarkers before administration, or by about at least 100% above the subject's level of the respective one or more energy biomarkers before administration.

When a decrease in a level of one or more energy biomarkers is desired to normalize the one or more energy biomarkers, the level of the one or more energy biomarkers can be decreased to a level within about at least two standard deviations of normal in a subject, more preferably decreased to within about at least one standard deviation of normal in a subject, decreased to within about at least one-half standard deviation of normal, or decreased to within about at least one-quarter standard deviation of normal, by administration of one or more compounds according to the invention. Alternatively, the level of the one or more energy biomarkers can be decreased by about at least 10% below the subject's level of the respective one or more energy biomarkers before administration, by about at least 20% below the subject's level of the respective one or more energy biomarkers before administration, by about at least 30% below the subject's level of the respective one or more energy biomarkers before administration, by about at least 40% below the subject's level of the respective one or more energy biomarkers before administration, by about at least 50% below the subject's level of the respective one or more energy biomarkers before administration, by about at least 75% below the subject's level of the respective one or more energy biomarkers before administration, or by about at least 90% below the subject's level of the respective one or more energy biomarkers before administration.

Enhancement of the level of one or more energy biomarkers is defined as changing the extant levels of one or more energy biomarkers in a subject to a level which provides beneficial or desired effects for the subject. For example, a person undergoing strenuous effort or prolonged vigorous physical activity, such as mountain climbing, could benefit from increased ATP levels or decreased lactate levels. As described above, normalization of energy biomarkers may not achieve the optimum state for a subject with a mitochondrial disease, and such subjects can also benefit from enhancement of energy biomarkers. Examples of subjects who could benefit from enhanced levels of one or more energy biomarkers include, but are not limited to, subjects undergoing strenuous or prolonged physical activity, subjects with chronic energy problems, or subjects with chronic respiratory problems. Such subjects include, but are not limited to, pregnant females, particularly pregnant females in labor; neonates, particularly premature neonates; subjects exposed to extreme environments, such as hot environments (temperatures routinely exceeding about 85-86 degrees Fahrenheit or about 30 degrees Celsius for about 4 hours daily or more), cold environments (temperatures routinely below about 32 degrees Fahrenheit or about 0 degrees Celsius for about 4 hours daily or more), or environments with lower-than-average oxygen content, higher-than-average carbon dioxide content, or higher-than-average levels of air pollution (airline travelers, flight attendants, subjects at elevated altitudes, subjects living in cities with lower-than-average air quality, subjects working in enclosed environments where air quality is degraded); subjects with lung diseases or lower-than-average lung capacity, such as tubercular patients, lung cancer patients, emphysema patients, and cystic fibrosis patients; subjects recovering from surgery or illness; elderly subjects, including elderly subjects experiencing decreased energy; subjects suffering from chronic fatigue, including chronic fatigue syndrome; subjects undergoing acute trauma; subjects in shock; subjects requiring acute oxygen administration; subjects requiring chronic oxygen administration; or other subjects with acute, chronic, or ongoing energy demands who can benefit from enhancement of energy biomarkers.

Accordingly, when an increase in a level of one or more energy biomarkers is beneficial to a subject, enhancement of the one or more energy biomarkers can involve increasing the level of the respective energy biomarker or energy biomarkers to about at least one-quarter standard deviation above normal, about at least one-half standard deviation above normal, about at least one standard deviation above normal, or about at least two standard deviations above normal. Alternatively, the level of the one or more energy biomarkers can be increased by about at least 10% above the subject's level of the respective one or more energy biomarkers before enhancement, by about at least 20% above the subject's level of the respective one or more energy biomarkers before enhancement, by about at least 30% above the subject's level of the respective one or more energy biomarkers before enhancement, by about at least 40% above the subject's level of the respective one or more energy biomarkers before enhancement, by about at least 50% above the subject's level of the respective one or more energy biomarkers before enhancement, by about at least 75% above the subject's level of the respective one or more energy biomarkers before enhancement, or by about at least 100% above the subject's level of the respective one or more energy biomarkers before enhancement.

When a decrease in a level of one or more energy biomarkers is desired to enhance one or more energy biomarkers, the level of the one or more energy biomarkers can be decreased by an amount of about at least one-quarter standard deviation of normal in a subject, decreased by about at least one-half standard deviation of normal in a subject, decreased by about at least one standard deviation of normal in a subject, or decreased by about at least two standard deviations of normal in a subject. Alternatively, the level of the one or more energy biomarkers can be decreased by about at least 10% below the subject's level of the respective one or more energy biomarkers before enhancement, by about at least 20% below the subject's level of the respective one or more energy biomarkers before enhancement, by about at least 30% below the subject's level of the respective one or more energy biomarkers before enhancement, by about at least 40% below the subject's level of the respective one or more energy biomarkers before enhancement, by about at least 50% below the subject's level of the respective one or more energy biomarkers before enhancement, by about at least 75% below the subject's level of the respective one or more energy biomarkers before enhancement, or by about at least 90% below the subject's level of the respective one or more energy biomarkers before enhancement.

### Use of Compounds in Research Applications, Experimental Systems, and Assays

The compounds of the invention can also be used in research applications, such as in vitro, in vivo, or ex vivo experiments in order to modulate one or more energy biomarkers in an experimental system. Such experimental systems can be cell samples, tissue samples, cell components or mixtures of cell components, partial organs, whole organs, or organisms. Such research applications can include, but are not limited to, use as assay reagents, elucidation of biochemical pathways, or evaluation of the effects of other agents on the metabolic state of the experimental system in the presence/absence of one or more compounds of the invention.

Additionally, the compounds of the invention can be used in biochemical tests or assays. Such tests can include incubation of one or more compounds of the invention with a tissue or cell sample from a subject to evaluate a subject's potential response (or the response of a specific subset of subjects) to administration of said one or more compounds, or to determine which compound of the invention produces the optimum effect in a specific subject or subset of subjects. One such test or assay would involve 1) obtaining a cell sample or tissue sample from a subject or set of subjects in which modulation of one or more energy biomarkers can be assayed; 2) administering one or more compounds of the invention to the cell sample(s) or tissue sample(s); and 3) determining the amount of modulation of the one or more energy biomarkers after administration of the one or more compounds, compared to the status of the energy biomarker prior to administration of the one or more compounds. Another such test or assay would involve 1) obtaining a cell sample or tissue sample from a subject or set of subjects in which modulation of one or more energy biomarkers can be assayed; 2) administering at least two compounds of the invention to the cell sample(s) or tissue sample(s); 3) determining the amount of modulation of the one or more energy biomarkers after administration of the at least two compounds, compared to the status of the energy biomarker prior to administration of the at least two compounds, and 4) selecting a compound for use in treatment, suppression, or modulation based on the amount of modulation determined in step 3).

### Formulations and Administration

The compositions, as described above, can be prepared as a medicinal preparation or in various other media, such as foods for humans or animals, including medical foods and dietary supplements. A “medical food” is a product that is intended for the specific dietary management of a disease or condition for which distinctive nutritional requirements exist. By way of example, but not limitation, medical foods may include vitamin and mineral formulations fed through a feeding tube (referred to as enteral administration). A “dietary supplement” shall mean a product that is intended to supplement the human diet and is typically provided in the form of a pill, capsule, and tablet or like formulation. By way of example, but not limitation, a dietary supplement may include one or more of the following ingredients: vitamins, minerals, herbs, botanicals; amino acids, dietary substances intended to supplement the diet by increasing total dietary intake, and concentrates, metabolites, constituents, extracts or combinations of any of the foregoing. Dietary supplements may also be incorporated into food, including, but not limited to, food bars, beverages, powders, cereals, cooked foods, food additives and candies; or other functional foods designed to promote cerebral health or to prevent or halt the progression of a neurodegenerative disease involving mitochondrial dysfunction. If administered as a medicinal preparation, the composition can be administered, either as a prophylaxis or treatment, to a patient in any of a number of methods. The compositions may be administered alone or in combination with other pharmaceutical agents and can be combined with a physiologically acceptable carrier thereof. The effective amount and method of administration of the particular formulation can vary based on the individual subject, the stage of disease, and other factors evident to one skilled in the art. During the course of the treatment, the concentration of the subject compositions may be monitored to insure that the desired level is maintained. The subject compositions may be compounded with other physiologically acceptable materials which can be ingested including, but not limited to, foods.

The compounds described herein can be formulated as pharmaceutical compositions by formulation with additives such as pharmaceutically acceptable excipients, pharmaceutically acceptable carriers, and pharmaceutically acceptable vehicles. Suitable pharmaceutically acceptable excipients, carriers and vehicles include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), and “Remington: The Science and Practice of Pharmacy,” Lippincott Williams & Wilkins, Philadelphia, 20th edition (2003) and 21^{st} edition (2005), incorporated herein by reference.

A pharmaceutical composition can comprise a unit dose formulation, where the unit dose is a dose sufficient to have a therapeutic or suppressive effect or an amount effective to modulate, normalize, or enhance an energy biomarker. The unit dose may be sufficient as a single dose to have a therapeutic or suppressive effect or an amount effective to modulate, normalize, or enhance an energy biomarker. Alternatively, the unit dose may be a dose administered periodically in a course of treatment or suppression of a disorder, or to modulate, normalize, or enhance an energy biomarker.

Pharmaceutical compositions containing the compounds of the invention may be in any form suitable for the intended method of administration, including, for example, a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like. For parenteral administration, the carrier can also be an oily ester such as ethyl oleate, isopropyl myristate, and the like. Compositions of the present invention may also be in the form of microparticles, microcapsules, liposomal encapsulates, and the like, as well as combinations of any two or more thereof.

Time-release or controlled release delivery systems may be used, such as a diffusion controlled matrix system or an erodible system, as described for example in: Lee, “Diffusion-Controlled Matrix Systems”, pp. 155-198 and Ron and Langer, “Erodible Systems”, pp. 199-224, in “Treatise on Controlled Drug Delivery”, A. Kydonieus Ed., Marcel Dekker, Inc., New York 1992. The matrix may be, for example, a biodegradable material that can degrade spontaneously in situ and in vivo for, example, by hydrolysis or enzymatic cleavage, e.g., by proteases. The delivery system may be, for example, a naturally occurring or synthetic polymer or copolymer, for example in the form of a hydrogel. Exemplary polymers with cleavable linkages include polyesters, polyorthoesters, polyanhydrides, polysaccharides, poly(phosphoesters), polyamides, polyurethanes, poly(imidocarbonates) and poly(phosphazenes).

The compounds of the invention may be administered enterally, orally, parenterally, sublingually, by inhalation (e.g. as mists or sprays), rectally, or topically in dosage unit formulations containing conventional nontoxic pharmaceutically acceptable carriers, adjuvants, and vehicles as desired. For example, suitable modes of administration include oral, subcutaneous, transdermal, transmucosal, iontophoretic, intravenous, intraarterial, intramuscular, intraperitoneal, intranasal (e.g. via nasal mucosa), subdural, rectal, gastrointestinal, and the like, and directly to a specific or affected organ or tissue. For delivery to the central nervous system, spinal and epidural administration, or administration to cerebral ventricles, can be used. Topical administration may also involve the use of transdermal administration such as transdermal patches or iontophoresis devices. The term parenteral as used herein includes subcutaneous injections, intravenous, intramuscular, intrasternal injection, or infusion techniques. The compounds are mixed with pharmaceutically acceptable carriers, adjuvants, and vehicles appropriate for the desired route of administration. Oral administration is a preferred route of administration, and formulations suitable for oral administration are preferred formulations. The compounds described for use herein can be administered in solid form, in liquid form, in aerosol form, or in the form of tablets, pills, powder mixtures, capsules, granules, injectables, creams, solutions, suppositories, enemas, colonic irrigations, emulsions, dispersions, food premixes, and in other suitable forms. The compounds can also be administered in liposome formulations. The compounds can also be administered as prodrugs, where the prodrug undergoes transformation in the treated subject to a form which is therapeutically effective. Additional methods of administration are known in the art.

Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent, for example, as a solution in propylene glycol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols that are solid at room temperature but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

The compounds of the present invention can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.Y., p. 33 et seq (1976).

The invention also provides articles of manufacture and kits containing materials useful for treating, preventing or suppressing symptoms associated with mitochondrial diseases. The article of manufacture comprises a container with a label. Suitable containers include, for example, bottles, vials, and test tubes. The containers may be formed from a variety of materials such as glass or plastic. The container holds a composition having an active agent which is effective for treating, preventing or suppressing symptoms associated with mitochondrial diseases. The active agent in the composition is one or more of the compounds of the invention. The label on the container indicates that the composition is used for treating, preventing or suppressing symptoms associated with mitochondrial diseases, and may also indicate directions for either in vivo or in vitro use, such as those described above.

The invention also provides kits comprising any one or more of the compounds of the invention. In some embodiments, the kit of the invention comprises the container described above. In other embodiments, the kit of the invention comprises the container described above and a second container comprising a buffer. It may further include other materials desirable from a commercial and user standpoint, including other buffers, diluents, filters, needles, syringes, and package inserts with instructions for performing any methods described herein.

In other aspects, the kits may be used for any of the methods described herein, including, for example, to treat an individual with symptoms associated with a mitochondrial disorder, to prevent symptoms associated with a mitochondrial disorder, or to suppress symptoms associated with a mitochondrial disorder in an individual.

The amount of active ingredient that may be combined with the carrier materials to produce a single dosage form will vary depending upon the host to which the active ingredient is administered and the particular mode of administration. It will be understood, however, that the specific dose level for any particular patient will depend upon a variety of factors including the activity of the specific compound employed, the age, body weight, body area, body mass index (BMI), general health, sex, diet, time of administration, route of administration, rate of excretion, drug combination, and the type, progression, and severity of the particular disease undergoing therapy. The unit dosage chosen is usually fabricated and administered to provide a defined final concentration of drug in the blood, tissues, organs, or other targeted region of the body. The effective amount for a given situation can be readily determined by routine experimentation and is within the skill and judgment of the ordinary clinician.

The dose of a compound or compounds disclosed herein useful in performing the invention is not restricted but varies depending on, for example, the age of the subject and the degree of risk of developing arterial stiffening. Possible values are 120 μg for men and 90 μg for women. Benefits may be derived by selecting dosages higher than these values, particularly in population groups where vitamin K deficiencies are common, for instance among postmenopausal women. For example, suitable dosages may lie in the range 10 to 1000 μg, more preferably 50 to 500 μg, and most preferably 100 to 200 μg of one or more compounds disclosed herein. It may be advisable to provide dosage ranges as high as from 1 to 200 mg/day, preferably from 5 to 150 mg/day, and more preferably from 10 to 100 mg/day.

Examples of dosages which can be used are an effective amount of compounds of Formula I, Ia, Ib, Ic, or Id within the dosage range of about 0.1 μg/kg to about 300 mg/kg, or within about 1.0 μg/kg to about 40 mg/kg body weight, or within about 1.0 μg/kg to about 20 mg/kg body weight, or within about 1.0 μg/kg to about 10 mg/kg body weight, or within about 10.0 μg/kg to about 10 mg/kg body weight, or within about 100 μg/kg to about 10 mg/kg body weight, or within about 1.0 mg/kg to about 10 mg/kg body weight, or within about 10 mg/kg to about 100 mg/kg body weight, or within about 50 mg/kg to about 150 mg/kg body weight, or within about 100 mg/kg to about 200 mg/kg body weight, or within about 150 mg/kg to about 250 mg/kg body weight, or within about 200 mg/kg to about 300 mg/kg body weight, or within about 250 mg/kg to about 300 mg/kg body weight. Other dosages which can be used are about 0.01 mg/kg body weight, about 0.1 mg/kg body weight, about 1 mg/kg body weight, about 10 mg/kg body weight, about 20 mg/kg body weight, about 30 mg/kg body weight, about 40 mg/kg body weight, about 50 mg/kg body weight, about 75 mg/kg body weight, about 100 mg/kg body weight, about 125 mg/kg body weight, about 150 mg/kg body weight, about 175 mg/kg body weight, about 200 mg/kg body weight, about 225 mg/kg body weight, about 250 mg/kg body weight, about 275 mg/kg body weight, or about 300 mg/kg body weight. Compounds of the present invention may be administered in a single daily dose, or the total daily dosage may be administered in divided dosage of two, three or four times daily.

While the compounds of the invention can be administered as the sole active pharmaceutical agent, they can also be used in combination with one or more other agents used in the treatment or suppression of disorders. Representative agents useful in combination with the compounds of the invention for the treatment, prevention or suppression of mitochondrial diseases include, but are not limited to, Coenzyme Q, vitamin E, idebenone, MitoQ, vitamins, and antioxidant compounds.

When additional active agents are used in combination with the compounds of the present invention, the additional active agents may generally be employed in therapeutic amounts as indicated in the Physicians' Desk Reference (PDR) 53rd Edition (1999), which is incorporated herein by reference, or such therapeutically useful amounts as would be known to one of ordinary skill in the art.

The compounds of the invention and the other therapeutically active agents can be administered at the recommended maximum clinical dosage or at lower doses. Dosage levels of the active compounds in the compositions of the invention may be varied so as to obtain a desired therapeutic response depending on the route of administration, severity of the disease and the response of the patient. When administered in combination with other therapeutic agents, the therapeutic agents can be formulated as separate compositions that are given at the same time or different times, or the therapeutic agents can be given as a single composition.

The invention will be further understood by the following nonlimiting examples.

### EXAMPLES

### Example A

### Screening Compounds of the Invention in Human Dermal Fibroblasts from Friedreich's Ataxia Patients

Test samples and solvent controls were tested for their ability to rescue FRDA fibroblasts stressed by addition of L-buthionine-(S,R)-sulfoximine (BSO), as described in Jauslin et al., Hum. Mol. Genet. 11(24):3055 (2002), Jauslin et al., FASEB J. 17:1972-4 (2003), and International Patent Application WO 2004/003565. Human dermal fibroblasts from Friedreich's ataxia patients have been shown to be hypersensitive to inhibition of the de novo synthesis of glutathione (GSH) with L-buthionine-(S,R)-sulfoximine (BSO), a specific inhibitor of GSH synthetase (Jauslin et al., Hum. Mol. Genet. 11(24):3055 (2002)). This specific BSO-mediated cell death can be prevented by administration of antioxidants or molecules involved in the antioxidant pathway, such as alpha-tocopherol, selenium, or small molecule glutathione peroxidase mimetics. However, antioxidants differ in their potency, i.e. the concentration at which they are able to rescue BSO-stressed FRDA fibroblasts.

MEM (a medium enriched in amino acids and vitamins, catalog no. 1-31F24-I) and Medium 199 (M199, catalog no. 1-21F22-I) with Earle's Balanced Salts, without phenol red, were purchased from Bioconcept. Fetal Calf Serum was obtained from PAA Laboratories. Basic fibroblast growth factor and epidermal growth factor were purchased from PeproTech. Penicillin-streptomycin-glutamine mix, L-buthionine (S,R)-sulfoximine, and insulin from bovine pancreas were purchased from Sigma. Calcein AM was purchased from Molecular Probes. Cell culture medium was made by combining 125 ml M199 EBS, 50 ml Fetal Calf Serum, 100 U/ml penicillin, 100 μg/ml streptomycin, 2 mM glutamine, 10 μg/ml insulin, 10 ng/ml EGF, and 10 ng/ml bFGF; MEM EBS was added to make the volume up to 500 ml. A 10 mM BSO solution was prepared by dissolving 444 mg BSO in 200 ml of medium with subsequent filter-sterilization. During the course of the experiments, this solution was stored at +4° C. The cells were obtained from the Coriell Cell Repositories (Camden, N.J.; repository number GM04078) and grown in 10 cm tissue culture plates. Every third day, they were split at a 1:3 ratio.

The test samples were supplied in 1.5 ml glass vials. The compounds were diluted with DMSO, ethanol or PBS to result in a 5 mM stock solution. Once dissolved, they were stored at −20° C.

Test samples were screened according to the following protocol: A culture with FRDA fibroblasts was started from a 1 ml vial with approximately 500,000 cells stored in liquid nitrogen. Cells were propagated in 10 cm cell culture dishes by splitting every third day in a ratio of 1:3 until nine plates were available. Once confluent, fibroblasts were harvested. For 54 micro titer plates (96 well-MTP) a total of 14.3 million cells (passage eight) were re-suspended in 480 ml medium, corresponding to 100 μL medium with 3,000 cells/well. The remaining cells were distributed in 10 cm cell culture plates (500,000 cells/plate) for propagation. The plates were incubated overnight at 37° C. in a atmosphere with 95% humidity and 5% CO₂ to allow attachment of the cells to the culture plate.

MTP medium (243 μL) was added to a well of the microtiter plate. The test compounds were unfrozen and 7.5 μL of a 5 mM stock solution was dissolved in the well containing 243 μL medium, resulting in a 150 μM master solution. Serial dilutions from the master solution were made. The period between the single dilution steps was kept as short as possible (generally less than 1 second).

Plates were kept overnight in the cell culture incubator. The next day, 10 μL of a 10 mM BSO solution were added to the wells, resulting in a 1 mM final BSO concentration. Forty-eight hours later, three plates were examined under a phase-contrast microscope to verify that the cells in the 0% control (wells E1-H1) were clearly dead. The medium from all plates was discarded, and the remaining liquid was removed by gently tapping the plate inversed onto a paper towel.

100 μl of PBS containing 1.2 μM Calcein AM were then added to each well. The plates were incubated for 50-70 minutes at room temperature. After that time the PBS was discarded, the plate gently tapped on a paper towel and fluorescence (excitation/emission wavelengths of 485 nm and 525 nm, respectively) was read on a Gemini fluorescence reader. Data was imported into Microsoft Excel (EXCEL is a registered trademark of Microsoft Corporation for a spreadsheet program) and used to calculate the EC₅₀ concentration for each compound.

The compounds were tested three times, i.e., the experiment was performed three times, the passage number of the cells increasing by one with every repetition.

The solvents (DMSO, ethanol, PBS) neither had a detrimental effect on the viability of non-BSO treated cells nor did they have a beneficial influence on BSO-treated fibroblasts even at the highest concentration tested (1%). None of the compounds showed auto-fluorescence. The viability of non-BSO treated fibroblasts was set as 100%, and the viability of the BSO- and compound-treated cells was calculated as relative to this value.

Certain compounds of the present invention such as:
2-hexyl-3-methylnaphthalene-1,4-dione;
2-butyl-3-methylnaphthalene-1,4-dione;
2,3-dimethylnaphthalene-1,4-dione;
naphthalene-1,4-dione;
2,3-dimethoxynaphthalene-1,4-dione; and
2-methoxynaphthalene-1,4-dione;
   exhibited protection against FRDA with an EC₅₀ of less than about 100 nM.

### Example B

### Screening Compounds of the Invention in Fibroblasts from Huntington's Patients

Compounds of the invention were tested using the screen as described in Example A, but substituting FRDA cells with Huntington's cells obtained from the Coriell Cell Repositories (Camden, N.J.; repository number GM 04281). The compounds were tested for their ability to rescue human dermal fibroblasts from Huntington's patients from oxidative stress.

Certain compounds of the present invention such as:
2-hexyl-3-methylnaphthalene-1,4-dione;
2,3-dimethylnaphthalene-1,4-dione;
2,3-dimethoxynaphthalene-1,4-dione;
naphthalene-1,4-dione;
2-butyl-3-methylnaphthalene-1,4-dione; and
2-methoxynaphthalene-1,4-dione;
   exhibited protection against Huntington's with an EC₅₀ of less than about 150 nM.

### Example C

### Screening Compounds of the Invention in Fibroblasts from Leber's Hereditary Optic Neuropathy Patients

Compounds of the invention were screened as described in Example A, but substituting FRDA cells with Leber's Hereditary Optic Neuropathy (LHON) cells obtained from the Coriell Cell Repositories (Camden, N.J.; repository number GM03858). The compounds were tested for their ability to rescue human dermal fibroblasts from LHON patients from oxidative stress.

Certain compounds of the present invention such as:
2,3-dimethylnaphthalene-1,4-dione;
2,3-dimethoxynaphthalene-1,4-dione;
naphthalene-1,4-dione;
2-hexyl-3-methylnaphthalene-1,4-dione;
2-butyl-3-methylnaphthalene-1,4-dione; and
2-methoxynaphthalene-1,4-dione;
   exhibited protection against LHON with an EC₅₀ of less than about 150 nM.

### Example D

### Screening Compounds of the Invention in Fibroblasts from Parkinson's Disease Patients

Compounds of the invention were screened as described in Example A, but substituting FRDA cells with Parkinson's disease (PD) cells obtained from the Coriell Cell Repositories (Camden, N.J.; repository number AG20439). The compounds were tested for their ability to rescue human dermal fibroblasts from Parkinson's disease patients from oxidative stress.

Certain compounds of the present invention such as:
2-hexyl-3-methylnaphthalene-1,4-dione;
naphthalene-1,4-dione;
2-butyl-3-methylnaphthalene-1,4-dione; and
2,3-dimethylnaphthalene-1,4-dione
   exhibited protection against PD with an EC₅₀ of less than about 200 nM.

### Example E

### Screening Compounds of the Invention in Fibroblasts from CoQ10 Deficient Patients

Compounds of the invention were tested using a screen similar to the one described in Example A, but substituting FRDA cells with cells obtained from CoQ10 deficient patients harboring a CoQ2 mutation. The compounds were tested for their ability to rescue human dermal fibroblasts from CoQ10 deficient patients from oxidative stress.

Certain compounds of the present invention such as:
2,3-dimethylnaphthalene-1,4-dione;
2,3-dimethoxynaphthalene-1,4-dione;
2-hexyl-3-methylnaphthalene-1,4-dione;
2-butyl-3-methylnaphthalene-1,4-dione; and
2-methoxynaphthalene-1,4-dione
   exhibited protection against CoQ10 deficiency with an EC₅₀ of less than about 100 nM.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1. A method of treating, preventing or suppressing symptoms associated with a mitochondrial disorder or dysfunction, comprising administering to a subject an effective amount of one or more compounds of the Formula I: 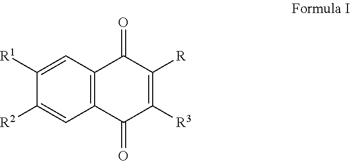
wherein,
R is selected from the group consisting of hydrogen, —O(C₁-C₆)alkyl, —(CH₂)₀₋₁₉—CH₃, —((CH₂)₂—CH(CH₃))₁₋₂₀—CH₃, 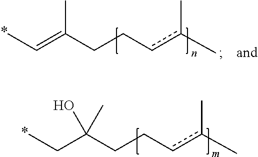
the * indicates the point of attachment to R;
the bond indicated by a dashed line is independently in each occurrence double or single and where each unit can be the same or different;
R¹, R² and R³ are independently of each other hydrogen, —(C₁-C₆)alkyl or —O(C₁-C₆)alkyl;
n is 0-12, wherein when n is 2-12 each unit can be the same or different; and
m is 1-12, wherein when m is 2-12 each unit can be the same or different;
with the proviso that when R¹ and R² are hydrogen, and R³ is —(C₁-C₆)alkyl, then R is not hydrogen or 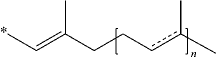
or any stereoisomer, mixture of stereoisomers, prodrug, metabolite, salt, crystalline form, non-crystalline form, hydrate or solvate thereof.

2. The method according to claim 1, wherein R is selected from the group consisting of:
—(CH₂)₀₋₁₉—CH₃, —((CH₂)₂—CH(CH₃))₁₋₂₀—CH₃; 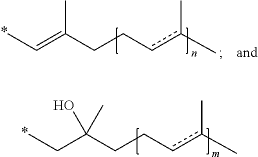
the * indicates the point of attachment to R;
the bond indicated by a dashed line is independently in each occurrence double or single;
R¹ and R² are independently of each other hydrogen, —(C₁-C₆)alkyl or —O(C₁-C₆)alkyl;
R³ is hydrogen or —(C₁-C₆)alkyl;
n is 0-12, wherein when n is 2-12 each unit can be the same or different; and
m is 1-12, wherein when m is 2-12 each unit can be the same or different;
or any stereoisomer, mixture of stereoisomers, salt, crystalline form, non-crystalline form, hydrate or solvate thereof.

3. The method according to claim 1, wherein R¹, R² and R³ are independently selected from —(C₁-C₆)alkyl.

4. The method according to claim 1, wherein R¹ and R² are hydrogen and R³ is —(C₁-C₆)alkyl.

5. The method according to claim 1, wherein R¹ and R² are independently of each other —O(C₁-C₆)alkyl and R³ is —(C₁-C₆)alkyl.

6. The method according to claim 1, wherein the one or more compound is a compound of Formula Ia: 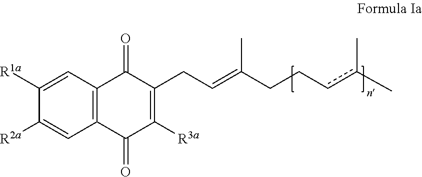
wherein,
the bond indicated by a dashed line is independently in each occurrence double or single, and where each unit can be the same or different;
R^{1a} and R^{2a} are independently of each other, —(C₁-C₆)alkyl or —O(C₁-C₆)alkyl;
R^{3a} is hydrogen or —(C₁-C₆)alkyl;
n′ is 0-12, wherein when n′ is 2-12 each unit can be the same or different;
or any stereoisomer, mixture of stereoisomers, salt, crystalline form, non-crystalline form, hydrate or solvate thereof.

7. The method according to claim 6, wherein R^{1a}, R^{2a} and R^{3a} are independently of each other —(C₁-C₆)alkyl.

8. The method according to claim 6, wherein R^{1a} and R^{2a} are —O(C₁-C₆)alkyl and R^{3a} is —(C₁-C₆)alkyl.

9. The method according to claim 6, wherein the bond indicated by a dashed line is a double bond.

10. The method according to claim 6, wherein the bond indicated by a dashed line is a single bond.

11. The method according to claim 1, wherein the one or more compounds of Formula I, are compounds of Formula Ib: 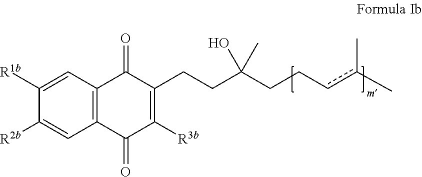
the bond indicated by a dashed line is independently in each occurrence double or single and where each unit can be the same or different;
R^{1b} and R^{2b} are independently of each other hydrogen, —(C₁-C₆)alkyl or —O(C₁-C₆)alkyl;
R^{3b} is hydrogen or —(C₁-C₆)alkyl;
m′ is 1-12, wherein when m′ is 2-12 each unit can be the same or different;
or any stereoisomer, mixture of stereoisomers, salt, crystalline form, non-crystalline form, hydrate or solvate thereof.

12. The method according to claim 11, wherein R^{1b}, R^{2b} and R^{3b} are independently of each other —(C₁-C₆)alkyl.

13. The method according to claim 11, wherein R^{1b} and R^{2b} are hydrogen and R^{3b} is —(C₁-C₆)alkyl.

14. The method according to claim 11, wherein R^{1b} and R^{2b} are —O(C₁-C₆)alkyl and R^{3b} is —(C₁-C₆)alkyl.

15. The method according to claim 11, wherein the bond indicated by a dashed line is a double bond.

16. The method according to claim 11, wherein the m′ is 3.

17. The method according to claim 11, wherein the compound is selected from:
2-(3-hydroxy-3,7,11,15-tetramethylhexadeca-6,10,14-trien-1-yl)-3-methylnaphthalene-1,4-dione;
2-(3-hydroxy-3,7,11,15-tetramethylhexadeca-6,10,14-trien-1-yl)naphthalene-1,4-dione; and any stereoisomer, mixture of stereoisomers, salt, crystalline form, non-crystalline form, hydrate or solvate thereof.

18. The method according to claim 11, wherein the bond indicated by a dashed line is a single bond.

19. The method of claim 1, wherein the one or more compound of Formula I are compounds of Formula Ic: 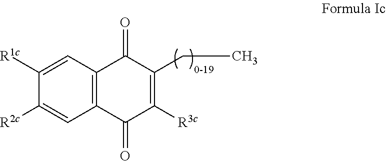
wherein,
R^{1c} and R^{2c} are independently of each other hydrogen, (C₁-C₆)alkyl or —O(C₁-C₆)alkyl;
R^{3c} is —(C₁-C₆)alkyl;
or any, salt, crystalline form, non-crystalline form, hydrate or solvate thereof.

20. The method according to claim 19, wherein R^{1c}, R^{2c} and R^{3c} are independently of each other —(C₁-C₆)alkyl.

21. The method according to claim 19, wherein R^{1c} and R^{2c} are hydrogen and R^{3c} is —(C₁-C₆)alkyl.

22. The method according to claim 19, wherein R^{1b} and R^{2c} are —O(C₁-C₆)alkyl and R^{3c} is —(C₁-C₆)alkyl.

23. The method according to claim 1, wherein the one or more compound of Formula I are compounds of Formula Id: 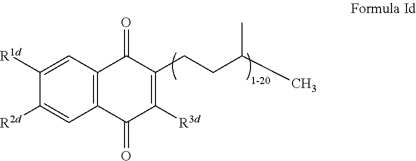
wherein,
R^{1d} and R^{2d} are independently of each other hydrogen, (C₁-C₆)alkyl or —O(C₁-C₆)alkyl;
R^{3d} is hydrogen or —(C₁-C₆)alkyl;
or any salt, crystalline form, non-crystalline form, hydrate or solvate thereof.

24. The method according to claim 23, wherein R^{1d}, R^{2d} and R^{3d} are independently of each other —(C₁-C₆)alkyl.

25. The method according to claim 23, wherein R^{1d} and R^{2d} are hydrogen and R^{3d} is —(C₁-C₆)alkyl.

26. The method according to claim 23, wherein R^{1d} and R^{2d} are —O(C₁-C₆)alkyl and R^{3d} is —(C₁-C₆)alkyl.

27. The method according to claim 1, additionally comprising a pharmaceutically acceptable excipient.

28. The method according to claim 1, wherein the mitochondrial disorder or dysfunction is selected from the group consisting of inherited mitochondrial diseases; Myoclonic Epilepsy with Ragged Red Fibers (MERRF); Mitochondrial Myopathy, Encephalopathy, Lactacidosis, Stroke (MELAS); Leber's Hereditary Optic Neuropathy (LHON); Dominant Optic atrophy (DOA); Leigh syndrome; Kearns-Sayre Syndrome (KSS); Friedreich's ataxia (FRDA); other myopathies; cardiomyopathy; encephalomyopathy; renal tubular acidosis; Parkinson's disease; Alzheimer's disease; amyotrophic lateral sclerosis (ALS); Huntington's Disease, developmental pervasive disorders or hearing loss.

29. The method according to claim 1, wherein the mitochondrial disorder or dysfunction is selected from the group consisting of inherited mitochondrial diseases; Myoclonic Epilepsy with Ragged Red Fibers (MERRF); Mitochondrial Myopathy, Encephalopathy, Lactacidosis, Stroke (MELAS); Leber's Hereditary Optic Neuropathy (LHON); Dominant Optic atrophy (DOA); Leigh syndrome; Kearns-Sayre Syndrome (KSS); and Friedreich's ataxia (FRDA).

30. The method according to claim 1, wherein the energy biomarker is selected from the group consisting of: lactic acid (lactate) levels, either in whole blood, plasma, cerebrospinal fluid, or cerebral ventricular fluid; pyruvic acid (pyruvate) levels, either in whole blood, plasma, cerebrospinal fluid, or cerebral ventricular fluid; lactate/pyruvate ratios, either in whole blood, plasma, cerebrospinal fluid, or cerebral ventricular fluid; phosphocreatine levels, NADH (NADH+H⁺) levels; NADPH (NADPH+H⁺) levels; NAD levels; NADP levels; ATP levels; reduced coenzyme Q (CoQ^{red}) levels; oxidized coenzyme Q (CoQ^{ox}) levels; total coenzyme Q (CoQ^{tot}) levels; oxidized cytochrome C levels; reduced cytochrome C levels; oxidized cytochrome C/reduced cytochrome C ratio; acetoacetate levels, .beta.-hydroxy butyrate levels, acetoacetate/.beta.-hydroxy butyrate ratio, 8-hydroxy-2′-deoxyguanosine (8-OHdG) levels; levels of reactive oxygen species; levels of oxygen consumption (VO2); levels of carbon dioxide output (VCO2); respiratory quotient (VCO2/VO2); exercise tolerance; and anaerobic threshold.
